(19) United States
(12) Patent Application Publication
(10) Pub. No.: US 2026/0224741 A1
(43) Pub. Date: Aug. 6, 2026

(54) METHOD OF DELIVERING NUCLEIC ACID TO MACROPHAGES AND/OR MONOCYTES AND COMPOSITIONS FOR USE THEREOF

(71) Applicant: NanoVation Therapeutics Inc., Vancouver (CA)

(72) Inventors: Jessica A.F.D. Silva, Vancouver (CA); Daniel Kurek, Vancouver (CA); Jayesh Kulkarni, Vancouver (CA)

(21) Appl. No.: 19/373,016

(22) Filed: Oct. 29, 2025

Related U.S. Application Data
(63) Continuation of application No. PCT/CA2025/050524, filed on Apr. 10, 2025.
(60) Provisional application No. 63/632,601, filed on Apr. 11, 2024.
(60) Provisional application No. 63/680,680, filed on Aug. 8, 2024.

(51) Int. Cl. A61K 48/00; A61K 39/395; C07K 16/28; C12N 15/88

(52) U.S. Cl. CPC A61K 48/0033; A61K 39/3955; A61K 48/005; C07K 16/2803; C12N 15/88

(57) ABSTRACT

The present disclosure provides a method for delivery of nucleic acid encoding a chimeric antigen receptor (CAR) for expression by a monocyte and/or macrophage to bind a receptor on a target cell or molecule in vivo, the method comprising contacting a lipid nanoparticle encapsulating the nucleic acid encoding the CAR with the immune cell ex vivo or in vivo, thereby causing cellular uptake of the nucleic acid to cause the immune cell to express the CAR. Further provided is a lipid nanoparticle for use in such method, the lipid nanoparticle having between 20 mol % and 70 mol % of a neutral or zwitterionic amphipathic lipid having a net-neutral charge at physiological pH, an ionizable cationic lipid, and optionally a sterol. The lipid nanoparticle is substantially uncharged at physiological pH and has an apparent pKa of between 6.0 and 7.5. The CAR provides a therapeutic, prophylactic or ameliorative effect in vivo.

This application is a bypass continuation of PCT Patent Application No. PCT/CA2025/050524 having an international filing date of Apr. 10, 2025, which claims the priority benefit of U.S. Provisional Patent Application No. 63/680,680 filed on Aug. 8, 2024, and U.S. Provisional Patent Application No. 63/632,601 filed on Apr. 11, 2024, which are each incorporated herein by reference in their entirety.

### TECHNICAL FIELD

The present disclosure relates to methods of delivery of nucleic acid to immune cells and compositions for delivery thereof.

### BACKGROUND

Immune cells are part of the body's natural defense against disease and thus the ability to modify and reprogram them presents an attractive modality to target a variety of disease conditions. For example, a new therapy, reliant on the genetic modification of immune cells to express receptors known as chimeric antigen receptor or “CAR” on their surface is a promising treatment option for various diseases or conditions, such as cancer, including certain hematological malignancies. For example, CAR-T therapy relies on transfecting T cells, typically ex vivo, to produce what are known as “CAR-T cells”. The CAR-T cells express chimeric antigen receptor (CAR) on their surface that directs them in vivo to receptors on target cells of interest (Atsavapranee et al., 2021, EBioMedicine, 67:103354). In the case of cancer, this includes a tumour-specific antigens expressed on a population of cancerous cells. Upon binding to the surface antigen, the CAR-T cells become activated and exert a desired therapeutic and/or prophylactic immune response against the target cell. While CAR-T cell therapy is approved for cancer, it is also being investigated for cardiac fibrosis or to treat autoimmune diseases (e.g., to reduce immune responses after transplants). A similar therapy is CAR-M, which involves transfecting macrophages and/or monocytes to express CAR on their surface to mount an immune response against a cellular target, such as tumours expressing a binding partner. An example is a CAR-M cell expressing an anti-HER2 CAR, which recruits the macrophages to cancer cells expressing HER2. Once at the tumour, CAR-M cells are activated to phagocytose the tumour cells expressing the antigen, leading to re-programming of the tumour microenvironment by releasing cytokines and/or drawing in other immune cells, such as T cells, natural killer cells and/or dendritic cells.

Current approaches used to transfect immune cells with nucleic acid encoding for CAR are reliant on viral vectors. However, viral vectors are limited by the amount of genetic material that they can carry and have safety concerns associated with permanent CAR expression (Billingsley et al., 2020, Nano Lett, 20 (3): 1578-1589). Electroporation is another method to deliver nucleic acid encoding for CAR to T cells. This involves introducing high voltage to make the cell membrane permeable for entry of the nucleic acid. However, the method can cause cell damage and requires the use of specialized equipment.

LNPs have been suggested as a next-generation mRNA-based modality for engineering CAR cells. Most LNPs clinically approved for nucleic acid delivery are based on a formulation known as Onpattro™. The Onpattro™ formulation is a lipid nanoparticle-based short interfering RNA (siRNA) drug formulation for the treatment of polyneuropathies induced by hereditary transthyretin amyloidosis. The success of this LNP delivery system paved the way for the clinical development of the leading LNP-based COVID-19 mRNA vaccines.

The Onpattro™ LNP formulation consists of four main lipid components, namely: ionizable amino lipid, distearoylphosphatidylcholine (DSPC), cholesterol, and polyethylene glycol conjugated lipids (PEG-lipids) at respective molar amounts of 50/10/38.5/1.5. Onpattro™ is still considered the gold standard for comparison in studies of LNP-mediated efficacy and current approaches to LNP design often make few deviations from the four-component system.

While strides have been made in research relating to LNP-mediated nucleic acid delivery, it is widely known that the Onpattro™ formulation largely accumulates in liver (hepatic) tissues. However, many immune cells reside in the lymphoid tissues, such as the lymph nodes, thymus, blood, spleen and bone marrow. To target immune cells, the ability of LNPs to be delivered in organs and tissues beyond the liver would greatly expand the clinical utility of therapeutics reliant on reprogramming immune cells. In order to improve the delivery of nucleic acid cargo to immune cell populations, the particles should exhibit enhanced circulation lifetimes. Traditional approaches to achieve this rely on optimizing the levels of PEG-lipid in the LNP, but the inclusion of PEG-lipids in LNPs often results in transfection potencies that are low or produce unfavorable immune responses. While serum stable, large unilamellar vesicles (LUV) used for small molecule delivery (e.g., anti-cancer drugs) have been prepared without PEG (Semple et al., 1996, Biochemistry, 35:2521-2525), its inclusion in solid-core type LNPs containing nucleic acid cargo is considered essential as it is thought to prevent aggregation of the particles during the formulation process (Kulkarni et al., 2020, Nanoscale, 12:23959-23966).

Recently, it has been found that LNPs encapsulating CAR significantly prolonged cell efficacy in vitro as a result of extended CAR-mRNA and CAR T cell persistence. (Kitte et al., 2023, Molecular Therapy, Methods & Clinical Development, 31:101139). However, a lower number of CAR molecules per T cell were observed on CAR T cells transfected with LNPs relative to those produced by electroporation.

There is a need in the art to improve the ex vivo or in vivo delivery of nucleic acid encoding nucleic acid to modify an immune cell, such as nucleic acid encoding CAR.

### SUMMARY

The present disclosure addresses one or more of the foregoing problems in the prior art and/or provides useful alternatives to known compositions for the delivery of cargo to immune cells to thereby produce modified immune cells to treat, prevent or ameliorate a disease or condition.

The present disclosure is based on the finding that an LNP formulation that includes elevated levels of neutral lipid, such as greater than 20 mol % of such lipid(s), exhibits unexpected improvements in the delivery of nucleic acid encoding CAR to immune cells relative to more conventional LNP formulations, such as an Onpattro™-type LNP (hereinafter referred to as a “baseline LNP”, “baseline formulation” or “baseline”) having 10 mol % DSPC. In some embodiments, populations of macrophages and/or monocytes transfected with CAR-LNPs described herein exhibit a higher rate of transfection in vivo relative to macrophages and/or monocyte populations transfected with the baseline LNP without elevated neutral lipid. In some embodiments, immune cells transfected with CAR-LNPs further exhibit improved activity against a target cell in vivo relative to cells transfected with the baseline LNP.

In some embodiments, populations of macrophages and/or monocytes transfected with CAR-LNPs described herein exhibit a higher rate of transfection in vivo relative to other immune cell populations, such as T cells.

The immune cell expressing the CAR, whether produced in vivo, ex vivo or in vitro, may be used for treatment or prevention of a wide range of diseases or conditions. In some embodiments, the LNPs produce modified immune cells that mount an immune response against a diseased cell.

According to one aspect of the disclosure, there is provided a method for delivery of nucleic acid encoding a chimeric antigen receptor (CAR) for expression by an immune cell selected from at least a monocyte or macrophage to bind a receptor on a target cell or molecule in vivo, the method comprising contacting a lipid nanoparticle encapsulating the nucleic acid encoding the CAR with the immune cell ex vivo or in vivo, thereby causing cellular uptake of the nucleic acid to cause the immune cell to express the CAR, the lipid nanoparticle having between 20 mol % and 70 mol % of a neutral or zwitterionic amphipathic lipid having a net-neutral charge at physiological pH, an ionizable cationic lipid, and optionally a sterol, wherein the lipid nanoparticle is substantially uncharged at physiological pH and has an apparent pKa of between 6.0 and 7.5, wherein the immune cell expressing the CAR provides a therapeutic, prophylactic or ameliorative effect in vivo.

According to one example of the foregoing aspect of the disclosure, the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

According to one example of the foregoing aspect or any embodiment thereof, the lipid nanoparticle comprises at least 25 mol % of the neutral or zwitterionic amphipathic lipid.

According to one example of the foregoing aspect or any embodiment thereof, the contacting is in vivo and the immune cell to which the CAR is delivered is present systemically in a subject.

According to one example of the foregoing aspect or any embodiment thereof, the contacting is in at least the blood, lymph nodes, spleen or bone marrow.

According to one example of the foregoing aspect or any embodiment thereof, the modified immune cell is part of a plurality of modified immune cells that include the macrophage or monocyte and one or more of a dendritic cell, T cell, neutrophil, basophil, eosinophil, mast cell, natural killer cell, myeloid cell, monocyte, lymphoid cell or a combination thereof.

According to one example of the foregoing aspect or any embodiment thereof, the plurality of modified immune cells comprises a T cell.

According to one example of the foregoing aspect or any embodiment thereof, the nucleic acid is mRNA or vector DNA for expressing an endogenous or exogenous protein, polypeptide or peptide in the immune cell.

According to one example of the foregoing aspect or any embodiment thereof, the lipid nanoparticle is for treating a disease or disorder that is an immunological disease or disorder.

According to one example of the foregoing aspect or any embodiment thereof, the lipid nanoparticle is for treating a disease or disorder that is a cancer.

According to one example of the foregoing aspect or any embodiment thereof, the cancer is a haematological cancer.

According to one example of the foregoing aspect or any embodiment thereof, the lipid nanoparticle is for reducing a B cell count in a blood compartment of the subject.

According to one example of the foregoing aspect or any embodiment thereof, lipid nanoparticle further produces a modified T cell that expresses the CAR.

According to a further aspect of the disclosure, there is provided a lipid nanoparticle comprising an encapsulated nucleic acid encoding a chimeric antigen receptor (CAR) for ex vivo or in vivo delivery to an immune cell that is at least a monocyte or macrophage to produce a modified immune cell expressing the CAR, the lipid nanoparticle having between 20 mol % and 70 mol % of a neutral lipid or zwitterionic amphipathic lipid having a neutral or net-neutral charge at physiological pH, an ionizable cationic lipid, and optionally a sterol, wherein the lipid nanoparticle is substantially uncharged at physiological pH and has an apparent pKa of between 6.0 and 7.5.

According to one example of the foregoing further aspect or any embodiment thereof, the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

According to one example of the foregoing further aspect or any embodiment thereof, the lipid nanoparticle is for contacting is in vivo and the immune cell to which the CAR is delivered is present systemically in a subject.

According to one example of the foregoing further aspect or any embodiment thereof, the lipid nanoparticle is for modifying the immune cell in vivo in a subject's blood, lymph nodes, spleen or bone marrow.

According to one example of the foregoing further aspect or any embodiment thereof, the lipid nanoparticle comprises at least 25 mol % of the neutral or zwitterionic amphipathic lipid.

According to one example of the foregoing further aspect or any embodiment thereof, the modified immune cell is part of a plurality of immune cells that include at least the macrophage or monocyte and one or more or a dendritic cell, T cell, neutrophil, basophil, eosinophil, mast cell, natural killer cell, myeloid cell, monocyte, lymphoid cell or a combination thereof.

According to a further aspect of the disclosure, there is provided a use of the lipid nanoparticle of the foregoing aspect or any embodiment thereof, to produce the modified immune cell having the therapeutic, prophylactic or ameliorative effect in vivo.

According to one embodiment, there is provided the use of the lipid nanoparticle as defined in the foregoing aspect or any embodiment thereof, which lipid nanoparticle is used to treat a disease or disorder that is a cancer.

According to one embodiment, there is provided the use of the lipid nanoparticle as defined in the foregoing aspect or any embodiment thereof in which the cancer is a haematological cancer.

According to one embodiment, there is provided the use of the lipid nanoparticle as defined in the foregoing aspect or any embodiment thereof in which the lipid nanoparticle is used to treat a disease or disorder that is an immunological disease or disorder.

According to one embodiment, there is provided the use of the lipid nanoparticle as defined in the foregoing aspect or any embodiment thereof for reducing a B cell count in a blood compartment of the subject.

According to a further aspect of the disclosure, there is provided an ex vivo immune cell preparation comprising the lipid nanoparticle as described in the foregoing aspect of any embodiment thereof.

In some embodiments, the immune cells in such preparation are obtained from a human subject. In some embodiments, the immune cells are introduced back to the human subject.

### BRIEF DESCRIPTION OF THE DRAWINGS

FIG. 1A depicts a CAR cell that has been transfected by the LNPs described herein binding to a target cell.

FIG. 1B shows an expression cassette for CAR mRNA cargo used in the Example section. The expression cassette in this example encodes for anti-CD19 targeting a CD19⁺ B cell and contains a Thy1.1 marker for assessing transfection.

FIG. 1C shows physicochemical characteristics, including polydispersity index (PDI), size (nm) and encapsulation % of CAR-LNPs A, B and C of Table 4 of Example 1 herein.

FIG. 2A, FIG. 2B and FIG. 2C show flow cytometry expression data for immune cell sub-types in the blood of mice administered CAR-LNPs A, B, and C of Table 4 of Example 1 at 1 mg/kg at a 24-hour endpoint. The cell types examined for transfection (Thy1.1 expression) were monocytes (FIG. 2A), T cells (FIG. 2B) and CD4/CD8 T cells (FIG. 2C).

FIG. 3A, FIG. 3B, FIG. 3C and FIG. 3D show flow cytometry data for immune cell sub-types in the bone marrow of mice administered CAR-LNPs A, B, and C of Table 4 of Example 1 at 1 mg/kg at a 24-hour endpoint. The cell types examined for transfection (Thy1.1 expression) were macrophages (FIG. 3A), T cells (FIG. 3B) and CD4/CD8 T cells (FIG. 3C). Transfection was assessed by measurement of the percentage of cells positive for Thy1.1. B cell populations were assessed by flow cytometry to determine their total percentage of alive cells (FIG. 3D). T

FIG. 4 shows physicochemical characteristics, including polydispersity index (PDI), size (nm) and encapsulation % of CAR-LNPs A, B, C, and D of Table 5 of Example 2 herein.

FIG. 5A, FIG. 5B, and FIG. 5C and FIG. 5D show flow cytometry data for immune cell sub-types in the blood of mice administered CAR-LNPs A, B, C and D of Table 5 of Example 2 at 24 hours post injection at various doses as indicated. The cell types examined for transfection (Thy1.1 expression) were monocytes (FIG. 5A), T cells (FIG. 5B) and CD4/CD8 T cells (FIG. 5C). Target cell depletion was assessed by measurement of the percentage of alive B cells by flow cytometry (FIG. 5D). For LNPs A and B (controls), the dose was 1 mg/kg and for LNPs C and D the doses were 0.3, 1.0 and 2.5 mg/kg.

FIG. 6A, FIG. 6B and FIG. 6C show flow cytometry data for immune cell sub-types in the blood of mice administered the CAR-LNPs A, B, C and D of Table 5 of Example 2. Transfection was assessed by measurement of the percentage of cells positive for Thy1.1. The experimental procedures and specific cell types examined are identical to FIGS. 5A-D above except measurements were taken at 48 hours post administration and the dose was 1 mg/kg (see also Example 3).

FIG. 7 shows physicochemical characteristics, including polydispersity index (PDI), size (nm) and encapsulation % of CAR-LNPs A and B of Table 6 of Example 4 herein.

FIG. 8A and FIG. 8B show the percentage and number of live B cells in the blood of mice administered phosphate buffered saline (PBS) and the CAR-LNPs A and B of Table 6 of Example 4. The LNP dose administered to mice was 0.1 mg/kg and blood samples were withdrawn at 6, 24, and 48 hours post administration (FIG. 8A). B cells counts (thousands (K)/μL) were graphed at 24 hours (FIG. 8B)

### DETAILED DESCRIPTION

### Nucleic Acid Encoding Chimeric Antigen Receptor (CAR)

In some embodiments, the LNP comprises a nucleic acid that encodes a chimeric antigen receptor (CAR).

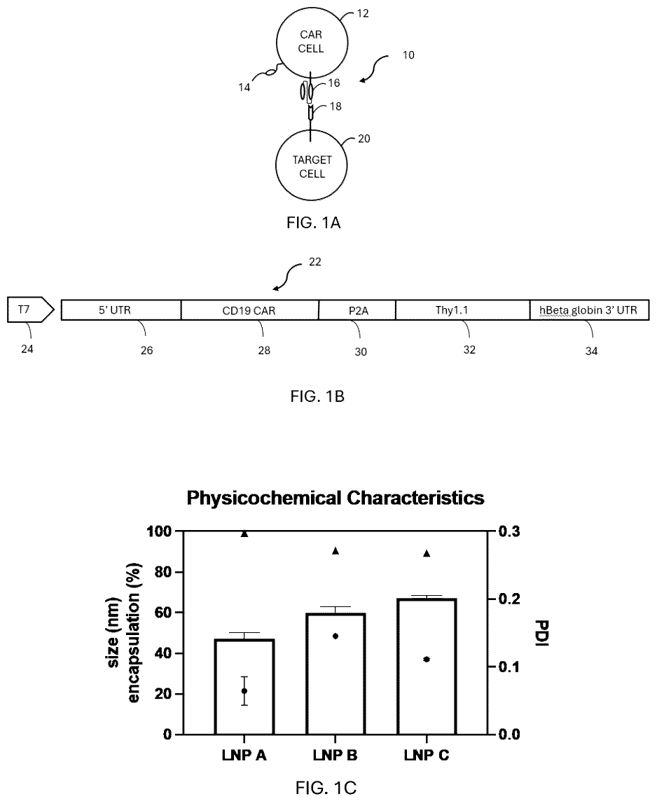

With reference to FIG. 1A, the nucleic acid encoding the CAR is delivered to an immune cell by the LNP having elevated neutral lipid content to produce a CAR cell 12. The CAR mRNA 16 is expressed on the surface of the CAR cell 12 and binds to a surface binding partner 18 of a target cell 20. In the examples herein, transfection efficiency of the immune cell was assessed by a marker 14 (Thy1.1) encoded by the CAR mRNA.

As used herein, the term “CAR” refers to a genetically engineered protein or polypeptide comprising an extracellular antigen recognition moiety, a transmembrane domain and one or more intracellular domains, in which the antigen recognition moiety is capable of recognizing a specific target antigen and modulating immune cell activity upon antigen binding.

As used herein, the term “CAR-M” refers to a CAR for expression in an immune cell selected from a macrophage and/or monocyte for enabling the immune cell to mediate an immune response, including but not limited to phagocytosis, antigen presentation and/or cytokine secretion.

As used herein, the term “CAR-T” refers to a CAR for expression in a T cell for enabling the T cell to mediate an immune response, including but not limited to cytotoxic activity, cytokine secretion and/or proliferation of immune cells.

As used herein, the term “nucleic acid encoding CAR,” includes any nucleic acid (e.g., RNA, DNA or hybrids thereof), that expresses the CAR (chimeric antigen receptor) and that is capable of encapsulation in the LNP herein. In one embodiment, an immune cell is modified by the LNP herein to produce a CAR immune cell.

In some embodiments, the CAR is encoded by a CAR construct, which is a nucleic acid that expresses at least an antigen recognition moiety, a transmembrane domain and an intracellular domain designed for CAR functionality. The CAR may be encoded by a CAR-M or CAR-T construct with similar domains but tailored for CAR-M or CAR-T therapy.

The nucleic acid that encodes the CAR is delivered via the LNP to the immune cell and results in expression of the CAR. In some embodiments, the immune cell is selected from at least one of a monocyte or macrophage, although additional immune cells may be transfected by the LNP, including but not limited to a T cell, dendritic cell, myeloid cell and/or a natural killer cell. The transfected CAR immune cell expresses chimeric antigen receptor (CAR) on its surface, which CAR targets the immune cell in vivo to receptors on target cells of interest. Upon binding to the surface antigen, the CAR immune cells become activated and exert a desired therapeutic and/or prophylactic immune response against the target cell. Without limitation, this may include destroying target cells through stimulated cell proliferation, cytotoxicity and/or increasing the secretion of factors that can affect other cells, including without limitation, cytokines, interleukins and/or growth factors. The use of LNPs with improved biodistribution in extrahepatic tissues/organs could be used to deliver mRNA encoding for the chimeric antigen receptor to a variety of target cells to treat a wide range of diseases or conditions, such as to treat or prevent a variety of diseases, including cancer, immunological disorders or cardiovascular conditions.

In one embodiment, the CAR is encoded by mRNA encapsulated by the lipid nanoparticle. The mRNA is delivered to an immune cell ex vivo or in vivo and subsequently expresses CAR and the CAR is inserted into the membrane of the immune cell. In another embodiment, the CAR is encoded by vector DNA.

In another embodiment, nucleic acid encoding CAR is introduced at one or more loci of a genome of the target cell by “knock-in”, and the CAR that is expressed from the one or more loci is subsequently inserted into the membrane of the immune cell. By way of example, a knock-in of a CAR nucleic acid may cause macrophages or monocytes to be more resistant to a tumour microenvironment and/or or improve their ability to engulf cancer cells. In another example, a CAR nucleic acid may be introduced at one or more loci using gene editing techniques (e.g., CRISPR), which are described in more detail below.

As noted above, the CAR includes an extracellular antigen recognition moiety and a transmembrane domain for insertion in the immune cell membrane. An internal domain or domains may be linked to the transmembrane domain or form part of the intracellular region of a transmembrane domain. As discussed below, such internal (intracellular) domain may include one or more signaling domains, which in some embodiments improve the ability of the immune cells to proliferate in vivo. In some embodiments, a linker is disposed between the antigen recognition moiety and the transmembrane domain.

### Antigen-Recognition Moiety

The antigen recognition moiety includes a variety of known structures for binding to an antigen of interest, typically a cell-surface antigen. In some embodiments, the antigen recognition moiety is an antibody fragment. For example, the antigen recognition moiety may include an antigen-recognizing single chain variable fragment (scFv) derived from an antibody sequence. The scFv may comprise a variable light (V_{L}) and variable heavy (V_{H}) regions of the scFv against the cell surface antigen of interest. The V_{L} and V_{H} chains may be connected by a linker region. A variety of linker regions may optionally link the V_{L} and V_{H} domains. In some non-limiting examples, the linker region comprises repeating glycine and serine residues.

In some embodiments, the V_{H} is located at the N terminus of the linker and the V_{L} is located at the C terminus of the linker. In other embodiments, the V_{L} is located at the N terminus of the linker and the VH is located at the C terminus of the linker. Alternatively, in some embodiments, the CAR antigen recognition moiety may include a single domain or multiple domains. Non-limiting examples of single domains include V_{HH} of camelid antibodies, natural ligands or artificial protein binding constructs. In some embodiments, bispecific and multispecific binding moieties that possess specificity to more than one target cell of interest are encompassed by the disclosure.

In some examples of the disclosure, the antigen recognition moiety comprises an scFv, a nanobody, a natural ligand, a peptide-based ligand, engineered proteins, an aptamer-based ligand, an Fc receptor-based ligand, a Megf10/MerTK-based ligand; a pattern recognition receptor (PRR)-based ligand or a synthetic non-immunoglobulin scaffold. The table below summarizes antigen-binding domain categories that can be used in CAR therapy.

TABLE 1
CAR antigen recognition moieties

| | Example | Therapeutic |
|---|---|---|
| Class | target | application |
| Single- | HER2 | Breast cancer |
| chain | CD19 | B cell |
| variable | | malignancies |
| fragment | | |
| (scFv) | | |
| Nanobody | EGFR | Solid tumours |
| (VHH) | B cell | (e.g., lung, |
| | maturation | colorectal |
| | antigen | and |
| | (BMCA) | glioblastoma) |
| | | Multiple |
| | | myeloma |
| Natural | CD47 | Solid tumours, |
| ligand- | (SIRPα- | hematologic |
| based | based | malignancies |
| | CAR-M) | |
| | NKG2D | |
| | ligands | |
| Peptide- | Integrin | Solid tumours |
| based | avβ6 | (e.g., pancreatic, |
| | PD-1- | head and neck |
| | based | and lung) |
| | CAR-T | Solid tumours |
| | (Targeting | (e.g., lung, |
| | PD-L1) | melanoma, |
| | | triple-negative |
| | | breast cancer) |
| Engineered | Anticalins | Breast or gastric |
| proteins | that | cancer |
| | target | |
| | HER2 | |
| | Lectin-like | |
| | domains | |
| | for | |
| | specificity | |
| | to glycan- | |
| | based | |
| | tumour | |
| | markers | |
| Aptamer- | CD44v6 | Solid tumours |
| based | (variant | (e.g., head and |
| | isoform of | neck and |
| | CD44) | pancreatic |
| | Protein | cancer) |
| | Tyrosine | Solid tumours |
| | Kinase 7 | (e.g., triple- |
| | (PTK7) | negative |
| | | breast cancer |
| | | or ovarian |
| | | cancer) |
| Fc | CD20 | B cell |
| receptor- | HER2 | malignancies |
| based | | (e.g., lymphoma |
| ligand | | and leukemia) |
| | | Breast or gastric |
| | | cancer |
| Pattern | Dectin-1 | Solid tumours or |
| recognition | (β-glucan | fungal infections |
| Receptor | on fungi- | Uses TLR2 |
| (PRR)- | infected | signaling to |
| based | or | activate T |
| | tumour | cells against |
| | cell) | tumours or |
| | PRR- | infected cells |
| | based | |
| | CAR | |
| | T TLR2 | |
| Synthetic | Designed | Cancer therapy |
| non-immuno- | Ankyrin | |
| globulin | Repeat | |
| scaffolds | Proteins | |
| | (DARPins) | |
| | targeting | |
| | vascular | |
| | endothelia | |
| | growth | |
| | factor | |
| | (VEGF) | |
| | and | |
| | hepatocyte | |
| | growth | |
| | factor | |
| | (HGF) | |

In one embodiment, the antigen recognition moiety is designed for CAR-M therapy. Some non-limiting examples include HER2 targeting scFv, which is used in HER2 CAR-M therapy; CD19-targeted scFv, which recognizes CD19, commonly expressed in B-cell malignancies; mesothelin-targeting scFv, which recognizes mesothelin overexpressed in mesotheliomas, ovarian and pancreatic cancers; epidermal growth factor receptor (EGFR)-targeting scFv, to target EGFR that is expressed in various solid tumours; and antigens targeting a programmed death-ligand 1 (PD-L1), which is an immune checkpoint molecule.

While scFvs are common antigen recognition moieties for CAR M therapies, alternative or modified antigen recognition moieties, such as those set forth in Table 1 above, may be employed in the practice of the disclosure. For example, a CAR-M may include FcRγ domains, which allow macrophages to recognize and engulf antibody-opsonized targets; Megf10- or MerTK-based receptors, which are used to recognize apoptotic cells or stressed tumour cells, triggering phagocytosis; or pattern recognition receptor (PRR)-based CARs having receptors that recognize tumour-specific glycoproteins or damage-associated molecular patterns (DAMPs).

In one embodiment, the antigen recognition moiety is designed for CAR-M therapy. In another embodiment, the antigen recognition moiety is designed for CAR-T therapy.

### Hinge Region

As noted above, the CAR in some embodiments includes a hinge (also referred to herein as a “spacer domain”) between the antigen recognition moiety and the transmembrane domain. Without being limited by theory, the hinge provides distance between the antigen recognition moiety and the surface of a cell membrane on which the CAR is expressed.

In some embodiments, the hinge is sufficiently flexible to facilitate binding of the antigen recognition moiety to the surface antigen on the target cell. As would be appreciated by those of skill in the art, a variety of hinge regions may be employed in the practice of the disclosure.

The hinge region may be an immunoglobulin-derived hinge. In some examples, the hinge is derived from IgG-like domain, such as Fc regions, including an IgG1, IgG2, IgG3, IgG4, IgA, IgD, IgE, IgM hinge or a fragment thereof. In certain embodiments, the Ig-based hinge is from a native immune cell molecule. An example is a hinge or spacer domain that is a hinge/spacer region of CD28. In another example, the hinge or spacer domain is from the hinge/spacer region of CD8 alpha. In another example, the IgG-based hinges are derived from IgG1, IgG2 or IgG4.

Optionally, the hinge further comprises human IgG-derived spacers consisting of CH2 and/or CH3 Ig-like domains. In one embodiment, the CH2 domain is removed or its function is ablated to improve target efficacy.

The disclosure also encompasses non-immunoglobulin hinges, such as hinges from natural receptors, such as a CD4 hinge or a CD3& hinge derived from a T cell receptor complex. In another embodiment, the hinge is an engineered or hybrid hinge, such as those designed to optimize flexibility without undesired interactions. This includes a hinge designed to minimize FcγR binding while maintaining flexibility or synthetic hinges designed for specific CAR applications.

The hinge may comprise proline-, glycine- and/or cysteine-rich sequences. In some embodiments, the proline introduces structural rigidity and facilitates an extended conformation. In another embodiment, the glycine increases flexibility, allowing the antigen recognition moiety to move freely. In a further embodiment, the cysteine forms a disulfide bond providing structural stability. In further embodiments, serine and threonine are found in glycosylated hinges, contributing to stability and reduced immunogenicity.

### Transmembrane Domain

The transmembrane domain resides in the bilayer of the immune cell and anchors the CAR in the immune cell membrane.

In some embodiments, the transmembrane domain is a hydrophobic alpha helix that spans the immune cell membrane. The transmembrane domain may function to facilitate molecular interactions between CARs. In some embodiments, the transmembrane domain may play a role in signal transduction. In some embodiments, the transmembrane domain may facilitate dimerization of CARs.

In some embodiments, the transmembrane domain is a single-span transmembrane domain and may be derived from CD4, CD8α, CD28. 4-1BB (CD137) or OX40 (CD134). Examples of transmembrane domains for CAR-M are provided in Table 2 below.

TABLE 2
Transmembrane (TM) domain for CAR-M applications

| | TM | |
|---|---|---|
| Application | domain | Properties |
| General CAR- | CD8α | Stable, neutral and |
| M therapy | | high surface expression |
| Enhanced | CD28 | Strong membrane |
| stability | | anchoring and supports |
| | | clustering |
| Long-term | 4-1BB | Improves receptor |
| CAR-M | | durability |
| function | | |

### Intracellular Domain

In certain embodiments, a CAR comprises one or more intracellular domains. In some embodiments, the one or more intracellular domains are or comprise a human intracellular domain, including a fragment thereof. In some embodiments, an intracellular domain may be a domain that is endogenous to a particular immune cell type (e.g., a modified immune cell as provided herein). In some embodiments, an intracellular domain may be a domain that is not endogenous to a particular immune cell type (e.g., a modified immune cell as provided herein). In some embodiments, an intracellular domain and/or other cytoplasmic domain of a CAR is responsible for activation of the cell in which the CAR is expressed (e.g., an immune cell). In some embodiments, an intracellular domain of a CAR is responsible for signal activation and/or transduction in an immune cell comprising the CAR.

In one embodiment, the CAR comprises at least a primary signaling domain. The intracellular domain may play a role in immune cell activation. For example, the signaling domain may enhance antibody dependent cellular phagocytosis, inflammatory signaling and antigen presentation. The intracellular domain may be designed for CAR-M or CAR-T therapy.

In those embodiments in which the CAR is designed for CAR-M therapy, the intracellular domain may include at least a phagocytic signaling domain. In some embodiments, the phagocytic signaling domain may include Fc Receptor-based domains (e.g., FcRγ, FcεRiγ and CD64). Fc receptor-based domains may be derived from Fc receptors that mediate antibody-dependent cellular phagocytosis. Such receptors may be used in CAR-M therapies to increase direct tumour engulfment. A further example is a Multiple EGF-like Domain 10 (Megf10), which is a phagocytic receptor involved in apoptotic cell clearance and may enhance non-opsonic phagocytosis.

In another example, the signaling domain is part of a tumour associated macrophage or “TAM” receptor family and may regulate phagocytosis and immune suppression in a tumour microenvironment. In some embodiments, this includes Fc Receptor Gamma Chain (FcRγ), which enhances phagocytosis and immune activation through ITAM signaling, DNAX-activating protein of 12 kDa (DAP12), which enhances macrophage activation and phagocytosis, CD137 or CD134, which are co-stimulatory receptors that enhance immune cell survival, cytokine production and activation, Signal Regulatory Protein Alpha (SIRPα) variants, which is an inhibitory receptor that interacts with CD47, wherein modified forms can block suppression; Toll-Like Receptors (TLR) Adapter Domains, involved in innate immune activation through MyD88 and TRIF signaling pathways.

In some embodiments, CAR-M intracellular domains are paired with an appropriate transmembrane domain. Non-limiting examples or such pairings are provided in Table 3 below:

TABLE 3
Examples of combinations of transmembrane ™ and
intracellular domains

| CAR-M | TM | Intracellular | |
|---|---|---|---|
| application | domain | domain | Function |
| General CAR- | CD8α | Megf10 or | Enhances |
| M therapy | | MerTK | phagocytosis |
| | | | without |
| | | | excessive |
| | | | inflammation |
| Strong | CD28 | FcRγ | Boosts |
| phagocytosis | | ITAM | engulfment |
| and activation | | | and immune |
| | | | activation |
| Pro-inflam- | 4-1BB | DAP12 | Enhances |
| matory and | | ITAM | cytokine |
| cytokine | | | production |
| release | | | and |
| | | | tumour |
| | | | clearance |
| Long-term | OX40 | DAP12 | Supports |
| CAR-M | | or FcRγ | durable |
| function | | | activation |

A pro-inflammatory and activation signaling domain may promote immune cell polarization toward a pro-inflammatory phenotype, such as to enhance anti-tumour activity.

In those embodiments in which the CAR is designed for CAR-T therapy, the intracellular domain may include a CD3ζ cytoplasmic domain. In some examples, the intracellular CD3ζ cytoplasmic domain has three immunoreceptor tyrosine-based activation motifs (ITAMs) which signal upon phosphorylation. Such CARs may possess enhanced immune cell function, and in some embodiments may include an additional protein molecule, causing production of cytokines or possess additional receptors such as costimulatory ligands. Examples of CARs known to those of skill in the art include TRUCKs (T cells Redirected for Universal Cytokine Killing) or armored CARs. (See Larson and Maus, 2021, Nat Rev Cancer, 21 (3): 145-161, which is incorporated herein by reference).

### Activation of CAR Immune Cells

The CAR immune cells are activated upon binding of the antigen recognition moiety to the antigen on the target cell. Such activation may cause clustering and/or immobilization of the CAR. The activation may be via direct CAR signaling, antibody-mediated activation, macrophage polarization and/or cross-talk with other immune cells, such as T cells.

In those embodiments in which activation is antigen-dependent (direct targeting), CAR-M cells are primarily activated when the CAR extracellular domain binds to a target antigen on the surface of target cells, such as tumour cells. This triggers intracellular signaling that activates the macrophage or monocyte. Activation of the macrophage or monocyte may promote phagocytosis, cytokine and chemokine secretion (e.g., IL-6, IL-12 and TNF-α) and/or proinflammatory polarization (M1 phenotype). Non-limiting examples of intracellular signaling pathways include MerTK/Megf10 pathway, which enhances engulfment of target cells; FcRγ or DAP12 ITAM signaling, which drives phagocytosis and inflammatory responses; and/or SYK/PI3K/AKT pathway, which activates macrophage-mediated immune responses.

In some embodiments, the activation is enhanced by antibody-mediated activation mechanisms. As would be understood by those of skill in the art, macrophages or monocytes may naturally express Fc receptors (e.g., FcγRI, FcγRIIA and FcγRIII), which bind to the Fc region of opsonizing antibodies. For example, when tumour cells are coated with therapeutic antibodies (e.g., trastuzumab for HER2+ cancers), CAR-M cells recognize and phagocytose these antibody-bound cells, even if they do not express the specific CAR antigen. In further embodiments, the presence of tumour-binding antibodies stimulates pro-inflammatory macrophage or monocyte activation, which may shift them to an M1-like phenotype (tumouricidal and pro-inflammatory). In some embodiments, such antibody-mediated activation may enhance tumour clearance beyond CAR-specific targeting.

In some embodiments, CAR-M therapy can work synergistically with existing monoclonal antibody treatments. For example, CAR-M therapy may be used in combination with Trastuzumab to enhance antibody-dependent cellular phagocytosis (ADCP) in HER2-expressing tumours; rituzimal in CD20+ B-cell lymphomas to improve macrophage-mediated tumour clearance; and cetuximab in EGFR+ cancers to increase FcR-mediated activation and antigen presentation.

In some embodiments, CAR-M therapy comprises inducing a macrophage polarization state that enhances immune activation. Without being bound by theory, macrophages or monocytes exist in a spectrum of functional states, with two primary polarization extremes, namely M1, which enhances immune activation and tumour destruction and M2, which promotes tissue repair and tumour progression. In some embodiments, a CAR-M cell is genetically modified to maintain an M1-like state to cause tumouricidal activity. In order to maintain activation in an M1-like state, CAR activation may occur via direct CAR activation by triggering intracellular signaling cascades to drive M1-like activation; overcoming tumour-induced M2 polarization by engineering CAR-M cells to be resistant to immunosuppressive signals caused by tumours (e.g., IL-10, TGF-β and CSF-1); and/or by enhancing adaptive immunity via antigen presentation by engulfing tumour cells and processing their antigens for MHC-II presentation, thereby activating cytotoxic T cells.

In some embodiments, CAR-M therapy comprises inducing cross talk between immune cells. In such embodiments, phagocytosed tumour cells are processed and their antigens are presented on MHC-II to T cells. This cross talk may bridge innate and adaptive immunity, stimulating a sustained anti-tumour T-cell response.

In those embodiments in which CAR T therapy is desired, CD3ζ chains may be present in the CAR, and phosphorylation of ITAM domains on the CD3ζ chain may initiate signaling through the tyrosine kinase-associated protein of 70 kDa (ZAP70). This initiates an effector response including proliferation, release of cytokines, metabolic alterations, and cytotoxicity.

In some embodiments, the activated CAR immune cells may exert a cytotoxic effect through secretion of granzyme and/or perforin. Alternatively, or additionally, death receptors are utilized, based on activation of downstream molecules such as BH3-interacting domain death agonist (BID) and FAS-associated death domain protein (FADD). As would be appreciated by those of skill in the art, cellular signaling from the internal domain or domains is dependent on the specific function of the domain chosen and can be modulated by introducing mutations thereof. The antigen recognition moiety may bind to an antigen on any target cell of interest. This may include an antigen on the surface of a tumour. Examples of target antigens on tumours include CD19, CD22 and B-cell maturation antigen (BMCA).

In some embodiments, the disease for treatment by CAR immune cells includes hematological malignancies, such as relapsed acute B cell leukemia, aggressive B cell lymphoma, 1, 2 and treatment-refractory multiple myeloma. In some embodiments, CAR immune cells can be used to treat solid tumors.

While CAR immune cell therapy in some embodiments is used to treat a cancer, other diseases and conditions may be treated by CAR immune cells. This includes, without limitation, cardiac fibrosis (see US 2023/02035338, incorporated herein by reference) or autoimmune diseases.

In some embodiments, the immune cell is modified to improve CAR T immune cell therapy. For example, gene editing of a T cell may be used to ameliorate CAR T cell dysfunction (e.g., T cell exhaustion), modulate cytokine production or knock in CAR cassettes at specific genomic locations.

### Nucleic Acid Encoding CAR

As used herein, the term “encapsulation,” with reference to incorporating the nucleic acid cargo within a lipid nanoparticle refers to any association of the nucleic acid with any lipid component or compartment of the lipid nanoparticle. In one example of the disclosure the nucleic acid is present in the core of the LNP.

The nucleic acid encoding the CAR includes, without limitation, RNA, including small nuclear RNA (snRNA), small nucleolar RNA (snoRNA), micro RNA (miRNA), guide RNA (gRNA), including single guide RNA (sgRNA), prime editing guide RNA (pegRNA), messenger RNA (mRNA), small activating RNA (saRNA), self-replicating RNA (srRNA), transamplifying RNA (taRNA), circular RNA (circRNA), long noncoding RNA (lncRNA), and transfer RNA (tRNA); and DNA such as vector DNA and linear DNA, or hybrids thereof. The nucleic acid length can vary and can include nucleic acid of 1-50,000 nucleotides in length. The nucleic acid can be in any form, including single stranded DNA or RNA, double stranded DNA or RNA, or hybrids thereof. Single stranded nucleic acid includes antisense oligonucleotides. The nucleic acid may be conjugated to another molecule, including a targeting moiety. An example of such a nucleic acid conjugate is an antibody-nucleic acid conjugate, or an oligosaccharide-nucleic acid conjugate, such as a GalNAc-nucleic acid conjugate.

As used herein, the term “messenger RNA” or “mRNA”, refers to a polynucleotide that encodes and expresses at least one protein, polypeptide or peptide. The term is meant to include mRNA that is circular or linear as well as small activating RNA (saRNA) and trans-amplifying RNA (taRNA).

The concentration of mRNA in the LNP may be between 0.01 and 20 mg/mL or between 0.01 and 10 mg/mL or between 0.05 and 5 mg/mL or between 0.075 and 4 mg/mL.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is chemically synthesized, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to formulate mRNAs of a variety of lengths. In some embodiments, the present disclosure may be used to formulate and encapsulate in vitro synthesized mRNA ranging from about 1-20 kb, about 1-20 kb, about 1-15 kb, about 1-10 kb, about 2-20 kb, about 2-15 kb, about 2-10 kb, about 3-20 kb, about 3-15 kb, about 3-10 kb, about 3-7 kb, about 5-20 kb, about 5-15 kb, about 5-12 kb, about 5-10 kb, about 8-20 kb, or about 8-15 kb in length.

In those embodiments in which the mRNA is linear, the synthesis includes the addition of a “cap” on the 5′ end, and a “tail” on the 3′ end. The presence of the cap provides resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In a further embodiment, the mRNA is circular. Advantageously, such mRNA lacks 5′ and 3′ ends and thus may be more stable in vivo due to its resistance to degradation by exonucleases. The circular mRNA may be prepared by any known method, including any one of the methods described in Deviatkin et al., 2023, “Cap-Independent Circular mRNA Translation Efficiency”, Vaccines, 11 (2), 238, which is incorporated herein by reference. Translation of the circular mRNA is carried out by a cap-independent initiation mechanism.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences are incorporated into mRNA for in vivo studies in animal models to assess biodistribution.

In another embodiment, the cargo is a DNA vector. The encapsulated DNA vector may be administered to a subject for the purpose of repairing, enhancing or blocking or reducing the expression of a cellular protein or peptide.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will most advantageously have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16 (11): 1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present disclosure can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Known procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

### Editing Cargo

A CAR may be introduced to an immune cell via editing cargo encapsulated in the LNP. In some embodiments, the editing cargo causes a knock-in of CAR.

As used herein, the term “editing cargo” includes a protein and/or nucleic acid-based system that causes modification of an immune cell at a specific locus or loci to produce a desired modification to treat, prevent or ameliorate a disease or condition.

As used herein, the term “nucleic acid editor” includes a protein and/or nucleic acid-based system that causes modification of any nucleic acid of an immune cell at a specific locus or loci to produce a desired modification to treat, prevent or ameliorate a disease or condition.

The cargo may comprise a nucleic acid that encodes for a protein or peptide that forms part of a nucleic acid editing complex. A “nucleic acid editing complex” includes without limitation protein and/or nucleic acid-based systems in which nucleic acid is inserted, deleted, modified (e.g., epigenetic editing) or replaced in the genetic material of an organism at a site-specific location.

The nucleic acid editing complex may be used for ex vivo or in vivo genetic modification of a T cell and includes post-translational modifications.

Alternatively or additionally, the cargo comprises a peptide or protein that is part of an editor or forms an editing complex.

The nucleic acid editing complex includes, without limitation, Cas-based (e.g., CRISPR or non-CRISPR), transcription activator-like effector nuclease (TALEN), megaTALs, zinc finger nuclease (ZFN), Adenosine Deaminase Acting on RNA (ADAR), prime editors, base editors, epigenetic, transposase, meganuclease, ARCUS gene editing systems or any variant or combination thereof. These nucleic acid editing systems are exemplary and include any cargo that can modify genetic material (including RNA transcripts and non-coding regions) of a cell to treat, prevent or ameliorate a disorder or disease. Without limitation, the gene editing system may include those that are designed by a process referred to as Directed Nuclease Editor (DNE), which is known to those of skill in the art.

Cas-based editing systems comprise CRISPR and non-CRISPR gene editing systems. In addition, the editing systems include those that cut DNA as well as epigenetic editing systems that modify nucleic acid markers, as discussed below.

The CRISPR gene editing cargo most advantageously comprises nucleic acid (e.g., mRNA) encoding for one or more of a Class II Cas nuclease family of proteins and a guide RNA. The nucleases encoded by the nucleic acid are enzymes with DNA endonuclease activity and can be directed to cleave a desired nucleic acid target by an appropriate guide RNA. The nuclease and guide RNA form a complex referred to as a ribonucleoprotein (RNP). In some embodiments, the nuclease is a Class II CRISPR enzyme, which is further subdivided into Types II, V and VI. According to one embodiment, the mRNA encodes for a Cas protein that is part of a Type II CRISPR/Cas system, such as a Cas9 protein or a Cpf1 protein.

In another embodiment, the mRNA encodes for a Cas protein that is part of a Type V CRISPR/Cas system, such as Cas12a. In another embodiment, the mRNA encodes for a Cas protein that is a Cas 13a, which is an RNA endonuclease and cleaves single-stranded RNA.

The guide RNA can direct the Cas nuclease to the target sequence on a target nucleic acid molecule, where the guide RNA hybridizes to the target sequence and the Cas nuclease cleaves or modulates the sequence. In some embodiments, the guide RNA binds to a class 2 nuclease, thereby providing specificity of cleavage.

Guide RNAs for the CRISPR/Cas9 nuclease system include CRISPR RNA (crRNA) or tracr RNA (tracr). In some embodiments, the crRNA can include a targeting sequence that is complementary to and hybridizes to a target sequence on a target nucleic acid molecule. The crRNA can also include a flagpole that is complementary to, and hybridize to, a portion of tracrRNA. In some embodiments, the crRNA can correspond to the structure of a naturally-occurring crRNA transcribed from a bacterial CRISPR locus, wherein the targeting sequence acts as a spacer for the CRISPR/Cas9 system. The flagpole corresponds to the part of the repetitive sequence adjacent to the spacer above the CRISPR locus.

The guide RNA of the RNP can target any sequence of interest through the targeting sequence of crRNA. In some embodiments, the targeting sequence of the guide RNA and the target sequence on the target nucleic acid molecule may be 100% complementary. In other embodiments, the targeting sequence of the guide RNA and the target sequence on the target nucleic acid molecule can comprise at least one mismatch.

The length of the targeting sequence may depend on the RNP system and components used. For example, different Cas proteins from different bacterial species have various optimal targeting sequence lengths. Thus, the targeting sequences are: 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length can be included. In some embodiments, the targeting sequence can comprise a length of 18 to 24 nucleotides. In some embodiments, the targeting sequence can comprise 19-21 nucleotides in length. In some embodiments, the targeting sequence can comprise a length of 20 nucleotides.

In some embodiments, the editing system includes Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csbl11, Csb2, Csb3, Csx17, Csx14, Csx10, Csx16, CsaX, Csx3, Csx1, Csx15, Csf1, Csf2, Csf3, Csf4, homologs thereof, or modified versions thereof.

As noted, non-CRISPR, Cas-based gene editing systems are encompassed by embodiments of the disclosure as well. A Cas-based editing system may include a Cas enzyme fused to deaminase (Luo et al., 2020, Microbial Cell Factories, 19 (93), incorporated herein by reference). An example is a cytosine base editor or an adenine base editor produced by fusing endonuclease Cas to cytosine deaminase pmCDA1 or heterodimer adenine deaminase TadA-TadA. A further non-limiting example is Cas fused to reverse transcriptase (Mohr et al., 2018, Mol Cell., 72 (4): 700-714, incorporated herein by reference).

Fanzor is a eukaryotic RNA-guided endonuclease that could function as a gene editor. (See Saito et al., 2023, Nature 620:660-668, which is incorporated herein by reference). In some embodiments, Fanzor proteins use RNA as a guide to target DNA precisely and can be modified to edit a T cell using LNPs described herein. In some examples, the compact Fanzor systems may have the ability to facilitate more improved delivery than CRISPR-Cas systems.

In those embodiments in which the cargo is a TALEN, the cargo comprises a nucleic acid encoding a peptide having a Transcription activator-like (TAL) effector DNA binding domain, a fragment or a variant thereof. In an embodiment, the system comprises a nucleic acid encoding a peptide having nuclease activity, e.g., endonuclease activity. In an embodiment, the peptide having nuclease activity is a type-II restriction 1-like endonuclease, e.g., a Fokl endonuclease.

In those embodiments in which the cargo is a ZFN, the nucleic acid may encode a peptide having: a Zinc finger DNA binding domain, a fragment or a variant thereof; and/or nuclease activity, e.g., endonuclease activity. In an embodiment, the Zn finger binding domain comprises 1, 2, 3, 4, 5, 6, 7, 8 or more Zinc fingers. In an embodiment, the peptide having nuclease activity is a type-II restriction 1-like endonuclease, e.g., a Fokl endonuclease.

Adenosine Deaminase Acting on RNA (ADAR) is another editing cargo encompassed by embodiments of the disclosure that may be used for post-transcriptional modification of RNA. Examples include ADAR1 and ADAR2. ADAR1 may catalyze posttranscriptional deamination of C6 of adenosines in dsRNA, converting them to inosines (see Song et al., 2022, PMC, 13 (1): e1665, incorporated herein by reference).

Meganucleases are enzymes in the endonuclease family that may induce homologous recombination, generate mutations and alter reading frames. The meganuclease includes homing endonucleases that are intron or intein endonucleases. In one embodiment, the meganuclease is from the LAGLIDADG family, a GIY-YIG endonuclease, an HNH endonuclease, a His-Cys box endonuclease or a PD-(D/E) XK endonuclease. Meganucleases may be combined with components of other gene editing system. In one embodiment, a DNA binding domain from a transcription activator-like (TAL) effector is combined with a meganuclease to produce a “megaTAL”. In another embodiment, a meganuclease may be fused to a DNA end-processing enzyme to promote an error-prone non-homologous end joining.

ARCUS nuclease is a gene editing system based on I-CreI, which is a kind of homing endonuclease that evolved in the algae Chlamydomonas reinhardtii. In some embodiments, the nuclease can deactivate itself after gene editing, thereby reducing off-targeting. ARCUS nucleases in some embodiments can generate a unique cleavage site that is a four-base-pair, 3′ overhang and may be able to carry out gene insertion, gene excision, gene repair or a combination thereof.

Epigenetic editing is also encompassed by examples of the disclosure. Such editing of genetic material does not cut nucleic acid but rather alters epigenomic marks “adorning” DNA. Changing the epigenic signature of a T cell can serve to modify an epigenetic signature of the cell and change its transcriptional profile.

Further examples of effector proteins include DNA methyltransferase, a fragment (e.g., a biologically active fragment) or variant thereof (e.g, DNMT1, DNMT2 DNMT3A, DNMT3B, DNMT3L, or CpG methyltransferase (M. Sssl)); or a poly comb repressive complex or a component thereof, e.g., PRC1 or PRC2, or PR-DUB, or a fragment (e.g., biologically active fragment) or a variant thereof.

In an embodiment, the epigenetic editor comprises a molecule that modifies chromatin architecture and/or modifies a histone. In an embodiment, the epigenetic modulator is a molecule that modifies chromatin architecture, e.g., a SWI/SNF remodeling complex or a component thereof. In an embodiment, the epigenetic modulator is a molecule that modifies a histone, e.g., methylates and/or acetylates a histone, e.g., a histone modifying enzyme or a fragment (e.g., biologically active fragment) or a variant thereof, e.g., HMT, HDM, HAT, or HD AC.

### Structural Non-Cationic Lipid

The LNP generally includes one or more structural lipids, meaning an amphipathic lipid that allows for the formation of particles and generally bears no net charge at physiological pH (7.4). The term includes neutral as well as zwitterionic lipids that impart substantially no charge at physiological pH to the LNP and includes phospholipids. In alternative embodiments, the structural lipid is a non-cationic lipid.

As used herein “substantially no charge”, means a net surface charge of about zero, or near neutral at physiological pH, such as without limitation about-2.5 mV to about 2.5 mV, or −5 mV to about 5 mV

In some embodiments, the structural lipid is a phosphatidylcholine lipid (PC-lipid) selected from distearoylphosphatidylcholine (DSPC), dioleoylphosphatidylcholine (DOPC), dimyristoylphosphatidylcholine (DMPC), 1-palmitoyl-2-oleoyl-phosphatidylcholine (POPC) and dipalmitoyl-phosphatidylcholine (DPPC) and a sphingomyelin, include those with a phosphatidylcholine head group.

The structural, neutral, zwitterionic or non-cationic lipid content in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of helper lipid content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the phosphatidylcholine lipid content is from 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

In some embodiments, the phosphatidylcholine lipid content is primarily composed of DSPC or DMPC or primarily DSPC. In such embodiments, the mixture may have a DSPC content of at least 20, 30, 35, 40 or 45 mol % based on the total lipid content of the lipid nanoparticle with the balance of the phosphatidylcholine lipid content being another phosphatidylcholine lipid(s). In another embodiment, the phosphatidylcholine content is made up of at least 40 or 50 mol % DSPC relative to the total phosphatidylcholine content of the lipid nanoparticle.

In certain embodiments, the DSPC lipid content is from 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

The LNP may comprise additional lipids besides a neutral or structural lipid. For example, the LNP may comprise structural lipids that have a net positive or negative charge at physiological pH. Generally, as discussed below, such lipids may be present at less than 10 mol % or less than 5 mol %.

In alternative embodiments, the mixture may have a DMPC content of at least 20, 30, 35, 40 or 45 mol % based on the total lipid content of the lipid nanoparticle. In another embodiment, the phosphatidylcholine content is made up of at least 40 mol % DMPC relative to the total phosphatidylcholine content of the lipid nanoparticle.

In another embodiment the structural, neutral, zwitterionic or non-cationic lipid content of the lipid nanoparticle is composed of less than 20, 10, or 5 mol % of non-phosphatidylcholine lipids, such as DOPE (measured relative to total phosphatidylcholine, structural lipid or neutral lipid content).

In another embodiment the structural, neutral, zwitterionic or non-cationic lipid content of the lipid nanoparticle is composed of less than 20, 10, or 5 mol % of non-phosphatidylcholine lipids, such as POPC (measured relative to total phosphatidylcholine, structural lipid or neutral lipid content).

In some embodiments, the transition temperature of the structural, neutral, zwitterionic or non-cationic lipid, e.g., a phospholipid having a choline head group, is at least 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. or 38° C. Without intending to be limited by any particular theory, it is believed that fusion and agglomeration of lipid nanoparticles with no hydrophilic polymer lipid conjugate (or low levels thereof) during particle formation using the mixing method described herein could be avoided by selecting a structural, neutral, zwitterionic or non-cationic lipid that is in the gel phase rather than in the disordered liquid crystalline phase at room temperature and above. The inclusion of such structural, neutral, zwitterionic or non-cationic lipid in the lipid nanoparticle may also improve blood stability after injection.

The structural, neutral, zwitterionic or non-cationic lipid content is determined based on the total amount of lipid in the lipid nanoparticle, including the sterol.

### Ionizable Lipid

The LNP of the disclosure has an ionizable lipid. The ionizable lipid may be charged at low pH and have substantially no net charge at physiological pH. This allows for electrostatic interactions between the lipid and the negatively charged nucleic acid cargo during initial formulation. Since the ionizable lipid is near neutral at physiological pH, toxicity and renal clearance are reduced. After cellular uptake by endocytosis, the acidic environment of the endosome leads to an increase in the net positive charge of the ionizable amino lipids, which promotes fusion with the anionic lipids of the endosomal membrane and subsequent membrane destabilization and release of the nucleic acid-based therapeutics into the cytoplasm to exert their effects.

In some embodiments, the LNP has an apparent pKa of between 5.0 and 7.5, between 6.5 and 7.5 or between 6.6 and 7.3. The apparent pKa is measured using a 6-(p-Toluidino)-2-naphthalenesulfonic acid (TNS) assay adapted from previous studies from other groups (Shobaki et al., 2018, International Journal of Nanomedicine, 13:8395-8410; and Jayaraman et al., 2012, Angew. Chem Int. Ed., 51:8529-8533, which are incorporated herein by reference for the purposes of determining apparent pKa). According to the method, a series of buffers are prepared spanning a pH range of 2-11 in 0.5 pH unit increments consisting of 130 mM NaCl, 10 mM ammonium acetate, 10 mM 2-(N-morpholino) ethanesulfonic acid (MES), and 10 mM HEPES. 0.15-0.2 mM of the LNP. A solution of 0.06 mM of TNS is subsequently mixed with 175 μL of the LNP at each buffered pH in triplicate in a black, polysterene 96-well plate, to yield a final concentration of 6.25 and 6 μM of lipid and TNS in each well, respectively. Fluorescence is subsequently measured using an SpectraMax™ M5 microplate reader at λₑₓ=321 nm, λₑₘ=445 nm. The fluorescence is then plotted against pH using a sigmoidal curve fit through Prism™, in which the pKa is determined to be the pH value with 50% of maximal fluorescent intensity.

In some embodiments, it is desirable to include less than 50 mol % ionizable lipid. That is, the ionizable lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the ionizable lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

As used herein, the term “ionizable cationic lipid” refers to a lipid that, at a given pH, such as physiological pH, is in an electrostatically neutral form and that accepts protons, thereby becoming electrostatically positively charged, and for which the electrostatically neutral form has a calculated logarithm of the partition coefficient between water and 1-octanol (i.e., a cLogP) greater than 8. In some embodiments, the cationic lipid has a pKa that is between 5.0 and 8.0, 5.0 and 7.5 or between 6.0 and 7.5.

In some embodiments, the ionizable cationic lipid has an amino group. In another embodiment, the ionizable cationic lipid has a single amino group that is ionizable. In some embodiments, the ionizable cationic lipid comprises a protonatable tertiary amine (e.g., pH titratable) head group. Such lipids include, but are not limited to sulfur lipids, such as MF019 described herein and DODMA. Other lipids that may be used in the practice of the disclosure include MC3- and KC2-type lipids, which are well-known to those of skill in the art. In further embodiments, the ionizable lipid is selected from one or more lipids set forth in WO 2022/246555; WO 2022/246568; WO 2022/246571; WO 2023/147657; WO2022/155728; WO 2023/215989; WO 2024/065041; WO 2024/065042; WO 2024/130421; WO 2024/065043; and U.S. 2024/0294462, each incorporated herein by reference

In one embodiment, the ionizable cationic lipid has a protonatable amino head group; at least two lipophilic moieties, wherein the amino head group has a central nitrogen atom or carbon atom to which each of the two lipophilic moieties are directly bonded; each lipophilic chain has between 15 and 40 carbon atoms in total; and wherein the lipid has (i) a pKa of between 6 and 7.5; and (ii) a ClogP of at least 11.

Optionally, at least one of the lipophilic moieties bonded to the head group has a biodegradable group. In one non-limiting example, at least one of the lipophilic moieties has an ester group in any orientation and a sulfur atom. In one embodiment, the ionizable cationic lipid has a lipophilic moiety of the formula:

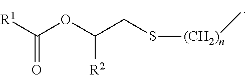

In one embodiment, R¹ and R² are, independently, linear, cyclic and/or branched optionally substituted C₃-C₂₀ alkyl and optionally with varying degrees of unsaturation; and n is 4 to 8.

In some embodiments, it is desirable to include less than 50 mol % ionizable cationic lipid in the LNP. That is, the ionizable cationic lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the ionizable cationic lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

The ionizable lipid component may include an ionizable anionic lipid as part of the ionizable lipid content. An example of such a lipid is cholesteryl hemisuccinate (CHEMS). Further examples of ionizable anionic lipids are described in co-pending and co-owned WO 2024/192528, which is incorporated herein by reference in its entirety.

In some embodiments, the ionizable cationic lipid is not a lipidoid structure, including but not limited to C12-200 (see Khare et al., 2022, AAPS Journal, 24:8, incorporated by reference) and related structures known to those of skill in the art.

### Sterol

The LNP further includes a sterol in some embodiments. The term “sterol” refers to a naturally-occurring or synthetic compound having a gonane skeleton and that has a hydroxyl moiety attached to one of its rings, typically the A-ring.

Examples of sterols include cholesterol, or a cholesterol derivative, the latter referring to a cholesterol molecule having a gonane structure and one or more additional functional groups.

The cholesterol derivative includes β-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N—(N′N′-dimethylaminoethyl) carbamoyl cholesterol (DC-Chol), 24 (S)-hydroxycholesterol, 25-hydroxycholesterol, 25 (R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

In one embodiment, the sterol is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is cholesterol and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is a cholesterol derivative and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In one embodiment, the combined (i) sterol content (e.g., cholesterol or cholesterol derivative thereof); and (ii) neutral lipid content is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

### Hydrophilic Polymer-Lipid Conjugate

In one embodiment, the lipid nanoparticle comprises a hydrophilic-polymer lipid conjugate capable of incorporation into the LNP. The conjugate includes a lipid or lipophilic moiety covalently attached to a polymer chain that is hydrophilic, optionally via a linker region. Examples of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate. The hydrophilic polymer lipid conjugate may also be a naturally-occurring or synthesized oligosaccharide-containing molecule, such as monosialoganglioside (GM1). The ability of a given hydrophilic-polymer lipid conjugate to enhance the circulation longevity of the LNPs herein could be readily determined by those of skill in the art using known methodologies.

The hydrophilic polymer lipid conjugate may be present in the nanoparticle at 0.5 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In certain embodiments, the hydrophilic polymer lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In another embodiment, the PEG-lipid conjugate is present in the nanoparticle at 0.5 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In one embodiment, the lipid nanoparticle has “substantially no hydrophilic polymer-lipid conjugate” or is “non-sterically stabilized”, “unshielded” or “uncoated”, meaning the lipid nanoparticle has less than 0.8 mol % total hydrophilic-polymer lipid conjugate content or other surface stabilizer content as measured based on the total lipid content of the nanoparticle as measured based on the total lipid content of the nanoparticle. In some embodiments, the hydrophilic-polymer lipid conjugate or other surface stabilizer content is less than 0.75, 0.70, 0.65, 0.60, 0.55, 0.50, 0.45, 0.40, 0.35, 0.30, 0.25, 0.20, 0.15, 0.10 mol % as measured based on the total lipid content of the nanoparticle. In further embodiments, the hydrophilic-polymer lipid conjugate or other surface stabilizer mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol % or 0 and 0.10 mol %.

As used herein, the term “surface stabilizer” is a macromolecule, including a protein, polysaccharide or polymer, including a block copolymer, that is used to stabilize a lipid nanoparticle, and in which at least a portion (e.g., hydrophilic) is present on the surface of the lipid nanoparticle. Such molecules are employed by those of skill in the art to prevent aggregation, improve shelf life and/or improve the stability of the particle after administration, such as the circulation lifetime of the lipid nanoparticle. The term includes surface stabilizers that are known to control the size of lipid nanoparticles, such as amphiphilic polymers (e.g., block co-polymer). As would be appreciated by those of skill in the art, a hydrophobic portion of the surface stabilizer may partition in a lipophilic portion of the lipid nanoparticle.

In some embodiments, the surface stabilizer is present at less than 0.75, 0.70, 0.65, 0.60, 0.55, 0.50, 0.45, 0.40, 0.35, 0.30, 0.25, 0.20, 0.15, 0.10 mol % as measured based on the total lipid content of the lipid nanoparticle. In further embodiments, the surface stabilizer mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol % or 0 and 0.10 mol %.

In some embodiments, the hydrophilic-polymer conjugate (e.g., a hydrophilic-polymer lipid conjugate) content is less than 0.75, 0.70, 0.65, 0.60, 0.55, 0.50, 0.45, 0.40, 0.35, 0.30, 0.25, 0.20, 0.15 or 0.10 mol % as measured based on the total lipid content of the nanoparticle. In further embodiments, the hydrophilic-polymer conjugate (e.g., a hydrophilic-polymer lipid conjugate) mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol %, 0 and 0.10 mol % or 0 and 0.05 mol %.

In some embodiments, the amphipathic polymer content is less than 0.75, 0.70, 0.65, 0.60, 0.55, 0.50, 0.45, 0.40, 0.35, 0.30, 0.25, 0.20, 0.15, 0.10 mol % as measured based on the total lipid content of the nanoparticle. In further embodiments, the amphipathic polymer mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol %, 0 and 0.10 mol % or 0 and 0.05 mol %. Examples of amphipathic polymers are provided in US 2021/0046192, which is incorporated herein by reference.

In some embodiments, the poloxamer content is less than 0.50, 0.45, 0.40, 0.35, 0.30, 0.25, 0.20, 0.15, 0.10 mol % as measured based on the total lipid content of the nanoparticle. In further embodiments, the poloxamer mol % content is between 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol %, 0 and 0.10 mol % or 0 and 0.05 mol %.

In further embodiments, the LNP lacks a surface stabilizer that is a protein, referred to as a protein stabilizer. This includes an apolipoprotein stabilizer, derivative or mimetic thereof (see e.g., WO 2023/233042, which is incorporated herein by reference). Such apolipoprotein may be selected from one or a combination of apo A1, apo A1-Milano, apo A2, apo A4, apo A5, apo B48, apo B100, apo C-1, apo C-1 I, apo C-111, apo C-IV, apo D, apo E, apo F, apo H, apo L and apo M. In some embodiments, the protein stabilizer content is less than 0.75, 0.70, 0.65, 0.60, 0.55, 0.50, 0.45, 0.40, 0.35, 0.30, 0.25, 0.20, 0.15 or 0.10 mol % as measured based on the total lipid content of the nanoparticle. In some embodiments, the protein stabilizer content is less than 0.45, 0.40, 0.35, 0.30, 0.25, 0.20, 0.15 or 0.10 mol % as measured based on the total lipid content of the nanoparticle. In further embodiments, the protein stabilizer content mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol % or 0 and 0.10 mol %. Since lipid nanoparticles can adsorb proteins after administration, the protein content is measured in vitro prior to administration.

Alternatively, in some embodiments a lipid nanoparticle preparation lacks or has low levels thereof of one or more stabilizing agents, which includes a surface stabilizer as described above and a cryoprotectant. In some embodiments, the lipid nanoparticle preparation has less than 2 w/v, 1.75 w/v, 1.50 w/v, 1.25 w/v, 1.00 w/v, 0.75 w/v, 0.50 w/v, 0.25, 0.10 or 0.05 w/v of one or more cryoprotectants in the preparation. In some embodiments, a lipid nanoparticle preparation having a plurality of LNPs has low levels or lacks glycerol and/or propylene glycol as a cryoprotectant, such as at concentration levels less than 2 w/v, 1.75 w/v, 1.50 w/v, 1.25 w/v, 1.00 w/v, 0.75 w/v, 0.50 w/v, 0.25, 0.10 or 0.05 w/v in the preparation.

In another embodiment, the lipid nanoparticle is “PEG-less”, meaning that the lipid nanoparticle has no detectable amounts of polyethylene-glycol lipid conjugate.

Examples of lipid nanoparticles with low levels or no hydrophilic polymer lipid conjugate or other surface stabilizer that can be used in the practice of the disclosure are described in co-owned and co-pending U.S. provisional patent applications 63/556,432 and 63/588,167, which are incorporated herein by reference. Such lipid nanoparticles are also described in Examples 1-3 herein.

### Additional Lipid Components

The LNP may comprise additional lipid components besides those described above (neutral lipid, cholesterol, ionizable cationic lipid and the optional hydrophilic polymer-lipid conjugate). Without limitation, such additional lipid components may be present at less than 10 mol %, 9 mol %, 8 mol %, 7 mol %, 6 mol %, 5 mol %, 4 mol %, 3 mol %, 2 mol %, 1 mol % or 0.5 mol % (relative to total lipid in the LNP). Such additional lipids include lipids comprising a targeting moiety, charged lipid (cationic or anionic lipid that is charged at physiological pH) or other lipid components such as vitamins (e.g., tocopherol). In some embodiments, the LNP consists essentially of neutral lipid, cholesterol, ionizable cationic lipid and the optional hydrophilic polymer-lipid conjugate, meaning any additional lipid is present at less than 5 mol % measured relative to total lipid in the LNP.

The LNP may comprise a targeting moiety for targeting the lipid nanoparticle to immune cells. An antibody conjugated LNP may be targeted to receptors present on immune cells, such as CD4 and CD8 present on T cells. The targeting moiety may be conjugated directly to a lipophilic moiety that resides in the LNP membrane or may be conjugated to the distal end of a hydrophilic polymer, if present. Examples of LNPs with targeting moieties are described in co-owned and co-pending WO 2024/119279, which is incorporated herein by reference.

In one embodiment, the LNP lacks a ligand-lipid conjugate for targeting to immune cells. In such embodiments, the ligand-lipid conjugate is undesirable as it may induce an immune response. Instead, targeting may be achieved by the inherent extrahepatic delivery properties of the lcLNP™ due to elevated phosphatidylcholine content. Thus, in some embodiments the ligand-lipid conjugate is present at less than 2 mol %, less than 1.5 mol %, less than 1 mol %, less than 0.5 mol %, less than 0.25 mol % or is 0 mol %.

In another embodiment, the additional component may include an anionic phospholipid, such as phosphatidylserine, and/or an ionizable anionic lipid. An example of such a lipid is cholesteryl hemisuccinate (CHEMS). Further examples of ionizable anionic lipids are described in co-pending and co-owned WO 2024/192528, which is incorporated herein by reference in its entirety.

Alternatively or additionally, the additional lipid component may include permanently charge cationic lipid, including lipids with a quarternary ammonium cation (e.g., DOTMA, DOSPA, DDAB and DOTAP) or a zwitterionic, anionic lipid, such as phosphatidylserine. Such permanently charged lipids, in some examples, are most advantageously present at less than 10 mol %, 9 mol %, 8 mol %, 7 mol %, 6 mol %, 5 mol %, 4 mol %, 3 mol %, 2 mol %, 1 mol %, 0.5 mol % or 0.25 mol % relative to total lipid content.

### Nanoparticle Preparation and Morphology

Delivery vehicles incorporating the cargo can be prepared using a variety of suitable methods, such as a rapid mixing/ethanol dilution process. Examples of preparation methods are described in Jeffs, L. B., et al., 2005, Pharm Res, 22 (3): 362-72; and Leung, A. K., et al., 2012, The Journal of Physical Chemistry. C, Nanomaterials and Interfaces, 116 (34): 18440-18450, each of which is incorporated herein by reference in its entirety.

Without being bound by theory, the mechanism whereby a lipid nanoparticle comprising encapsulated cargo can be formed using the rapid mixing/ethanol dilution process can be hypothesized as beginning with formation of a dense region of hydrophobic mRNA-ionizable lipid core at low pH (e.g., pH 4) surrounded by a monolayer of helper lipid/cholesterol that fuses with smaller empty vesicles as the pH is raised due to the conversion of the ionizable cationic lipid to the neutral form. As the proportion of bilayer helper lipid increases, the bilayer lipid progressively forms blebs and the ionizable lipid migrates to the interior hydrophobic core. At high enough helper lipid contents, the exterior bilayer preferring helper lipid can form a complete lipid layer, such as a continuous or discontinuous bilayer, around the interior trapped volume.

The LNP may comprise a “core” region. It has been observed that the LNP core is non-homogeneous in that it includes both an electron dense region and an aqueous portion or compartment as visualized by cryo-EM microscopy. In some embodiments, the core may be characterized as non-solid. Without being limiting, the electron dense region within the core may be partially surrounded by the aqueous portion or compartment within the enclosed space as observed by cryo-TEM. The aqueous portion may form a distinct aqueous region or compartment within the lipid nanoparticle. In other words, it is believed that the aqueous portion or compartment is not merely a hydration layer.

In one embodiment, at least about one fifth of the core (trapped volume) contains the aqueous portion or compartment, and in which the electron dense region within the core is partially contiguous with the lipid layer comprising the bilayer, as determined qualitatively by cryo-EM. In another embodiment, at least about one quarter of the core contains the aqueous portion or compartment, and in which the electron dense core is either partially contiguous with the lipid layer comprising the bilayer, as determined qualitatively by cryo-EM. In a further embodiment, at least about one third of the core contains the aqueous portion or compartment, and in which the electron dense region is either partially contiguous with the lipid layer comprising the bilayer, as determined qualitatively by cryo-EM. In another embodiment, at least about one half of the core contains the aqueous portion or compartment, and in which the electron dense core is partially contiguous with the lipid layer comprising the bilayer, as determined qualitatively by cryo-EM.

In another embodiment, the electron dense region of the LNP surprisingly appears to be completely surrounded by the aqueous portion of the core as visualized by cryo-TEM microscopy. This morphology is observed in a single plane and a portion of the electron dense region as observed is contiguous with the lipid layer (e.g., bilayer) but cannot be seen since this portion is not within the plane that can be visualized.

In one embodiment, the electron dense region is generally spherical in shape. In another embodiment, the electron dense region is hydrophobic.

The lipid nanoparticles herein may exhibit particularly high trapping efficiencies of mRNA. Thus, in one embodiment, the trapping efficiency is at least 50, 55, 60, 65, 70, 75, 80, 85 or 90%.

In another embodiment, the cargo and cationic ionizable lipid are present in the electron dense region. In a further embodiment, the helper lipid is present in the lipid layer comprising the bilayer.

The lipid nanoparticle may comprise a single bilayer or may be a combination of a bilayer and a monolayer in some embodiments. In one embodiment, the lipid layer is a continuous bilayer that surrounds the core.

In certain embodiments the electron dense region of the core is separated from the lipid layer comprising the bilayer by the aqueous portion or compartment. For example, the disclosure provides a lipid nanoparticle preparation comprising a plurality of lipid nanoparticles in which at least 20%, 30%, 40%, 50%, 60% or 70% of the particles as determined by cryo-EM microscopy have a core with an electron dense region and an aqueous portion or compartment and in which the aqueous portion or compartment is partially surrounded by the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have an elongate shape (e.g., generally oval-shaped) as determined qualitatively by cryo-EM microscopy. In this latter embodiment, the electron dense region of the core may be partially surrounded the aqueous space as visualized by cryo-EM microscopy.

In one embodiment, the lipid nanoparticle is part of a preparation of lipid nanoparticles, and wherein the electron dense region of at least 20% of the lipid nanoparticles are either (i) enveloped by the aqueous portion, or (ii) is partially surrounded by the aqueous portion and wherein a portion of a periphery of the electron dense region is contiguous with the lipid layer, as visualized by cryo-EM microscopy in a single plane.

In certain embodiments, the disclosure provides a lipid nanoparticle preparation comprising a plurality of lipid nanoparticles in which generally at least 10%, 20%, 30%, 40%, 50%, 60% or 70% of the particles as determined by cryo-EM microscopy have a core with an electron dense region that is contiguous with the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have a core comprising an electron dense region that appears to be surrounded or enveloped by a continuous aqueous space disposed between the lipid layer (e.g., bilayer) and the electron dense region, as visualized in one plane by cryo-EM microscopy.

LNPs are visualized by cryo-TEM as described in co-owned and co-pending WO 2022/251959, incorporated herein by reference.

In another embodiment, the polydispersity index (PDI) of the LNP preparation is less than 0.50, 0.45, 0.40, 0.35, 0.30, 0.25, 0.2, 0.15, 0.12 or 0.10.

In another embodiment, the particle size distribution is such that at least 90% of the particles in the LNP preparation of the disclosure have a diameter of between 40 and 150 nm or between 40 and 140 nm or between 45 and 150 nm or between 50 and 150 nm or between 50 and 120 nm or between 50 and 140 nm.

The lipid nanoparticles herein may exhibit particularly high encapsulation efficiencies of nucleic acid. As used herein, the term “encapsulation,” with reference to incorporating the cargo (e.g., nucleic acid) within a lipid nanoparticle refers to any association of the cargo with any lipid component or compartment of the lipid nanoparticle, including a lipophilic or the aqueous portion. In one embodiment, the cargo is present at least in the core of the LNP.

In one embodiment, the encapsulation efficiency is at least 50, 55, 60, 65, 70, 75, 80, 85, 90% or 92%. The encapsulation efficiency of the cargo is determined as set forth in the Materials and Methods section in the Examples herein.

Embodiments of the present disclosure also provide lipid nanoparticles described according to the molar ratio between the positively charged amine groups of the amine lipid (N) and the negatively charged phosphate groups (P) of the oligonucleotide to be encapsulated. This may be mathematically represented by the equation N/P. In one embodiment, the N/P ratio of the lipid nanoparticle is between 2 and 15, between 3 and 15, between 4 and 15 or between 4.5 and 10 or between 5 and 10 or between 5.5 and 8.

In one embodiment, the N/P ratio of the lipid nanoparticle is at least 2, 3, 4, 4.25, 4.50, 4.75, 5.0, 5.25, 5.5, 5.75, 6.0 or 6.25. The upper limit may be 15, 14, 13, 12, 11, 10, 9 or 8. The disclosure also encompasses a combination of any two of the upper and lower limits.

In one embodiment, the lipid nanoparticle has a weight nucleic acid/micromole of total lipid that is 0.05:1 to 1:1. In one embodiment, the lower limit is 0.06:1, 0.08:1, 0.10:1, 0.12:1, 0.14:1, 0.16:1, 0.18:1, 0.20:1, 0.22:1, 0.24:1, 0.26:1, 0.28:1, 0.30:1, 0.32:1, 0.34:1, 0.36:1, 0.38:1 or 0.40:1 weight nucleic acid/micromole of total lipid. In another embodiment, the upper limit is 0.80:1, 0.82:1, 0.84:1, 0.86:1, 0.88:1, 0.90:1, 0.92:1, 0.94:1, 0.96:1 or 0.98:1 weight nucleic acid/micromole of total lipid. The disclosure also encompasses a combination of any two of the upper and lower limits.

In one embodiment, the mRNA copy number/LNP is 1-10 or 4-8.

### Improved Delivery of Nucleic Acid to Immune Cells

It has also been shown herein that the lipid nanoparticle compositions exhibit improved delivery of nucleic acid encoding CAR to a macrophage and/or monocyte cell in vivo.

As used herein, “expression” of nucleic acid (e.g., mRNA or pDNA) refers to translation of the nucleic acid into a peptide (e.g., an antigen), polypeptide, or protein and also can include, as indicated by context, the post-translational modification of the peptide, polypeptide or fully assembled protein (e.g., enzyme). The nucleic acid (e.g., mRNA or pDNA) may encode for a protein or peptide in a gene editing complex. The polypeptide or protein encoded by the nucleic acid may comprise one or more functional domains, optionally linked by spacer regions.

In one embodiment, the lipid nanoparticle exhibits at least a 5%, 10%, 30% or 40% increase in gene expression of a CAR nucleic acid in vivo as measured in a macrophage and/or monocyte relative to baseline Onpattro-type LNP control.

As used herein an “Onpattro-type” or “baseline formulation” or “LNP control” has ionizable lipid 1 (IL1): distearoylphosphatidylcholine (DSPC): cholesterol: PEG₂₀₀₀-dimyristoylglyceride (PEG₂₀₀₀-DMG) at 50:10:38.5:1.5 as set forth in Example 1. The ionizable cationic lipid 1 is nor-MC3 as set forth in WO 2022/246571 and reproduced below:

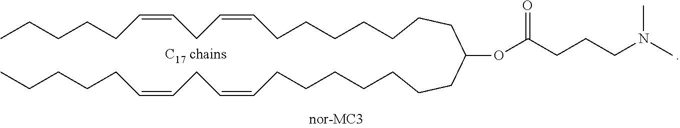 [SMILES: CCCCC/C=C\C/C=C\CCCCCCCC(CCCCCCC/C=C\C/C=C\CCCCC)OC(=O)CCCN(C)C]

The CAR-LNP of the disclosure may provide improved in vivo nucleic acid CAR expression in macrophages and/or monocytes than the baseline formulation. Whether or not a lipid nanoparticle exhibits such enhanced CAR expression in a macrophage and/or monocyte sub-population can be determined by flow cytometry studies in an in vivo mouse model. In such embodiments, a CAR construct with a Thy1.1 marker is used to detect nucleic acid CAR expression in a macrophage or monocyte cell population. In particular, according to such embodiments, LNP mRNA systems are prepared encapsulating mRNA coding for CAR and CAR expression in cell populations in macrophage and/or monocyte cells are evaluated using the blood and/or spleen flow cytometry following systemic administration. It will be understood that measurement of CAR expression using Thy1.1 is used as a proxy to assess whether a given LNP falls within the scope of the disclosure. Any clinical formulations meeting these expression criteria will typically not contain a reporter, although flow cytometry animal studies based on Thy1.1 expression as described herein can be used to determine whether or not a lipid nanoparticle possesses the improved macrophage and/or monocyte cell targeting. In other words, the test using an animal model to assess improved delivery to a macrophage and/or monocyte relative to a baseline LNP, will typically involve obtaining a clinical LNP formulation with a CAR cargo having a therapeutic or prophylactic effect on a subject (typically human) and conducting tests on an LNP having the same lipid components to determine if the LNP formulation itself exhibits improved macrophage and/or monocyte targeting quantified as described above. For example, to determine if a given clinical LNP formulation exhibits the improved macrophage and/or monocyte cell delivery, the therapeutic CAR cargo could be replaced with mRNA coding for CAR and a reporter and expression in vivo in an animal model (rather than a human) compared to the baseline formulation.

To assess whether a given lipid nanoparticle exhibits an increase in expression of CAR in a relevant immune cell at 4 hours, 12 hours, 24 hours, 48 hours or 3 days post-injection, the CAR-LNP of the disclosure is compared to the baseline formulation of Example 1. The two LNPs being compared are subjected to the same experimental methods and materials to determine in vivo expression as set forth in the Materials and Methods in the Example section herein.

In one embodiment, the lipid nanoparticle exhibits at least a 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290% or 300% increase in expression of mRNA encoding CAR as measured in vivo in a macrophage and/or monocyte population sub-set at 24 hours and/or 48 hours post-injection as compared to a lipid nanoparticle encapsulating the same cargo with a baseline formulation of nor-MC3/DSPC/cholesterol/PEG-lipid at 50/10/38.5/1.5; mol:mol, wherein the protein expression from the nucleic acid is measured in a mouse by detection of a Thy1.1 marker translated from the mRNA. The measurement is carried out using flow cytometry and protein expression from the nucleic acid as determined by quantifying % positive cell for the expressed marker in a given immune cell subset as set forth in Example 1 in the blood or bone marrow. The percentage increase is determined by comparing the percentage positive cells (i.e., detection of Thy1.1) in a given macrophage and/or monocyte cell population in the bone marrow or blood and comparing this percentage to the percentage of cells positive for the marker resulting from injecting the baseline formulation using otherwise identical materials and methods.

The efficacy of the CAR modified immune cell transfected by the LNP of the disclosure can be assessed by depletion of B cells using flow cytometry as described in the Examples.

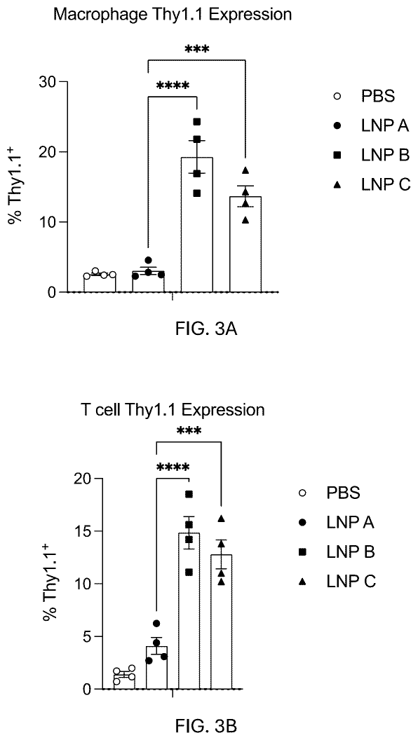

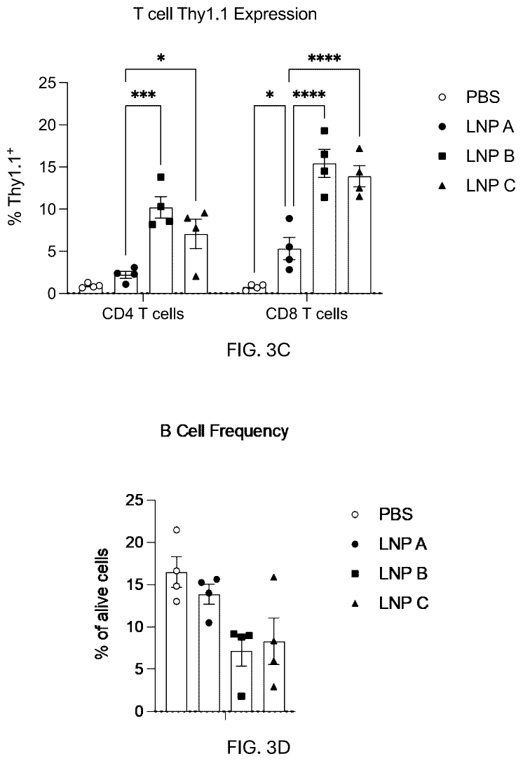

In one embodiment, the lipid nanoparticle of the disclosure and encapsulating the construct of FIG. 1B causes at least a 2%, 5% or 10% decrease the B cell population frequency as a percentage of total alive cells in the blood of a mouse as measured in vivo at 24 hours post-injection as compared to a lipid nanoparticle encapsulating the same cargo with a baseline formulation of nor-MC3/DSPC/cholesterol/PEG-lipid at 50/10/38.5/1.5; mol:mol, wherein the B cell population is measured in a mouse by detection of percentage of B cells of total alive cells in the blood a mouse by flow cytometry as described in the Materials and Methods in the Example section (e.g., depicted in FIG. 3D).

### Modified Immune Cells Expressing a CAR to Provide a Therapeutic, Prophylactic or Ameliorative Effect In Vivo

The LNPs of the disclosure may be used to transfection macrophages and/or monocytes as well as a variety of other immune cells as described below.

In particularly advantageous embodiments, the immune cell is a “macrophage”, which is an immune cell that is capable of engulfing substances, whether self or non-self, through phagocytosis. Such substances include, without limitation, cellular debris, apoptotic cells, pathogens, foreign particles and other extracellular materials. Typically, the macrophage recognizes a CAR antigen recognition moiety and becomes activated. In some embodiments, the macrophage targeted by the CAR resides in a tissue of the subject. In some embodiments, the macrophage secretes cytokines, presents antigens and/or contributes to tissue homeostasis and repair.

In some embodiments, the macrophages form a component of the innate immune system. Macrophages may phagocytose pathogens or tumour cells. In some embodiments, macrophages are capable of one or more of the following functions: phagocytosis of dead and dying cells, microorganisms, cancer cells, cellular debris, or other foreign substances; cytotoxicity against certain target cells (e.g., tumour cells); and/or presentation of antigens (e.g., tumour antigens) to cause an adaptive immune response.

Macrophages may be present in the tumour microenvironment of numerous cancers and are often referred to as tumour-associated macrophages (TAMs). A phenotype characterized as an M2-like TAM, contributes to the general suppression of anti-tumour immune responses. It has been found that TAMs can be “reprogrammed” via pro-inflammatory signals, thereby switching them from an M2 phenotype to a more M1 phenotype is associated with productive anti-tumour immune responses. Inducing endogenous TAMs to switch to M1-type cells and engineering macrophages that cannot be subverted into M2 would greatly enhance anti-tumour immunotherapy and represent a significant advance in the field.

In further particularly advantageous embodiments, the immune cell is a monocyte, which is an immune cell that has the ability to differentiate into a macrophage or dendritic cell, such as upon migration into a tissue. Typically, monocytes are capable of recognizing and responding to a self or non-self substance. In some embodiments, monocytes carry out immune surveillance, inflammatory responses and/or tissue homeostasis through phagocytosis, antigen presentation and/or cytokine secretion.

The LNP encoding a nucleic acid for expression of CAR may also be used to transfect T cells and/or natural killer (NK) cells. In some embodiments, the LNP transfects both a macrophage and/or monocyte and additionally other immune cells, such as a T cell.

A T cell has T cell receptors that enable antigen recognition and activation thereof. In some embodiments, a T cell mediates immune functions such as cytotoxic activity, cytokine secretion and/or interaction with other immune cells.

Natural Killer (NK) cells are cytotoxic lymphocytes that form part of the innate immune system. In some embodiments, NK cells are characterized as having CD56 and lacking CD3 (termed “CD56+, CD3−”). Without being limiting, unlike most immune cells that detect antigen based on presented MHC-I on cell surfaces, NK cells can recognize and kill stressed cells without the presence of antibodies and/or MHC. In some embodiments, this may allow for a more rapid immune response than for other activated immune cells. In some embodiments, NK cells induce the death of tumour cells even in the absence of surface adhesion molecules and antigenic peptides.

NK cells can be engineered to express CAR that recognize surface antigens of target cells (e.g. virus infected cells and cancer cells) in combination with a signaling molecule to activate immune cells. This modification can counteract inhibitory receptors, thereby enhancing NK cells' specific killing effect on target cells. Without being limited by theory, CAR-NK cells may have a limited lifetime in circulation, thereby reducing the risk of side effects such as graft-versus-host-disease and side effects on normal tissues. CAR-NK cells can be used to treat cancers, viruses or other disease conditions.

In further embodiments, the LNP additionally transfects one or more of dendritic cells, neutrophils, basophils, eosinophils, mast cells, myeloid cells and/or lymphocytes.

### Administration of LNPs and Pharmaceutical Formulations Thereof.

In some embodiments, the lipid nanoparticle comprising nucleic acid is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In some embodiments, the LNP is contacted with a cell either ex vivo or in vivo. As used herein, the term “contacting” or “to contact” means establishing a physical interaction between the lipid nanoparticle either in vivo, in vitro or ex vivo using methods that are well known in the art. In one embodiment, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. In some embodiments, more than one type of cell may be contacted by a lipid nanoparticle composition.

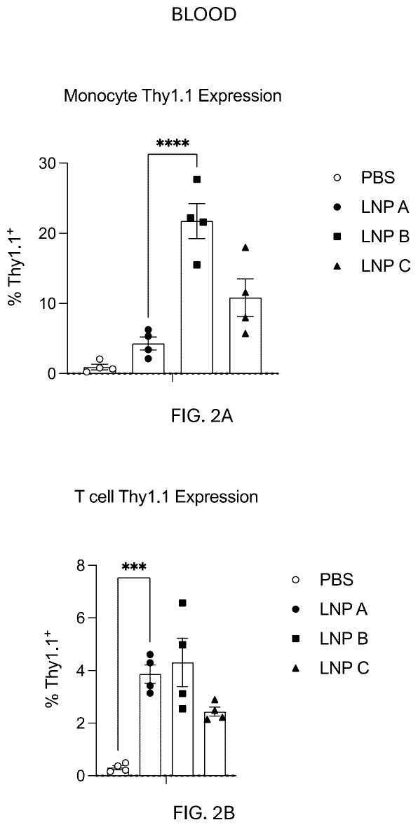

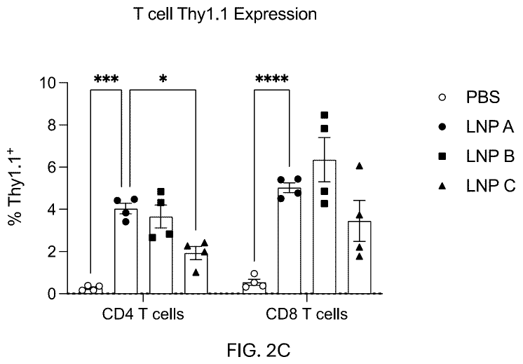

In one embodiment, the LNPs herein are injected intravenously to a subject and are targeted to extrahepatic organs or tissues, including the vascular system (e.g., blood). As discussed, the inventors have found that LNPs with elevated levels of a neutral or zwitterionic lipid (e.g., a phosphatidylcholine lipid or other structural lipid) have improved extrahepatic biodistribution. In particular, flow cytometry has revealed that macrophages and monocytes have increased expression of CAR mRNA as measured by a reporter construct in the blood relative to a baseline formulation at 24 hours post-administration (FIG. 2A).

In some embodiments, the LNPs comprise nucleic acid encoding CAR for transfecting an immune cell in vivo or ex vivo so as to modify the immune cell to express the CAR, which modified immune cell expressing the CAR reduces B cell count in a subject. In some embodiments, the immune cells so modified are for treating a subject in which it is desired to reduce B cell count. Non-limiting examples of diseases or conditions that can be treated to reduce B cell count include cancers such as B cell malignancies or B-cell mediated autoimmune diseases, such as systemic lupus, neuromyelitis optica spectrum disorder, myasthenia gravis and multiple sclerosis. According to such embodiments, the reduction of B cells, including depletion thereof, may at least partially restore normal immune function.

In some embodiments, the CAR is designed to target CD19, which is a B-cell surface antigen. In such embodiments, the CAR may include an antigen recognition moiety derived from an anti-CD19 monoclonal antibody and wherein the transmembrane domain has one or more intracellular signaling domains, such as CD3ζ and/or co-stimulatory domains (e.g., CD28, and/or 4-1BB), wherein the CAR enables genetically modified immune cells, including but not limited to macrophages, monocytes and/or T cells, among other immune cells, to recognize and eliminate CD19 expressing cells through cytotoxic activity, cytokine secretion and/or immune modulation.

In another embodiment, the CAR cells are produced ex vivo. In such embodiments, immune cells are obtained from a subject (e.g., patient) or a donor thereof, contacted with the LNP herein to produce the CAR cells and then administered to the subject. The CAR cells can be derived autologously from immune cells in a subject's own blood or allogeneically from a donor. The immune cells obtained from the subject or donor may be selected from monocytes, macrophages, T cells or a combination thereof. In some embodiments, leukocytes are obtained by leukocyte apheresis and peripheral blood mononuclear cells are separated and collected. In some embodiments, certain populations of immune cells are stimulated to proliferate and after proliferation and purification, the cells are contacted with the LNP ex vivo.

In some embodiments of the disclosure, the LNP is administered to a subject and immune cells are modified in vivo after administration. Such embodiment is advantageous as it avoids isolating immune cells from the subject or a donor. Improved targeting of the LNP to extrahepatic tissues or organs advantageously provides improvements in delivery to immune cell populations that reside in, for example, blood, spleen, bone marrow and/or the lymphatic system. In one embodiment, the LNP is part of a pharmaceutical composition administered parenterally, i.e., intra-arterially, intravenously, subcutaneously or intramuscularly. In yet a further embodiment, the pharmaceutical compositions are for intraosseous injection (IO).

The pharmaceutical composition provides a preventative, therapeutic or ameliorative effect and comprises pharmaceutically acceptable salts and/or excipients.

The composition described herein may be administered to a subject such as a patient. The term subject as used herein includes a human or a non-human subject. In one embodiment, the subject is a primate, which includes a human or non-human subject.

The examples below are intended to illustrate the preparation of specific lipid nanoparticle nucleic acid preparations and properties thereof but are in no way intended to limit the scope of the invention.

The examples are intended to illustrate the preparation of specific lipid nanoparticle preparations and properties thereof but are in no way intended to limit the scope of the invention.

The article “a” or “an” as used herein is meant to include both singular and plural, unless otherwise indicated.

### EXAMPLES

### Materials and Methods

### LNP Preparation

The LNPs were prepared by dissolving mRNA in 25 mM sodium acetate, pH 4.0, while the lipid components at the mole % specified were dissolved in absolute ethanol. The lipids in ethanol and the mRNA in buffer were combined in a 1:3 volume by volume ratio using a t-junction with dual-syringe pumps. The solutions were pushed through the t-junction at a combined flow rate of 20 mL/min (5 mL/minute for the lipid-containing syringe, 15 mL/minute for the mRNA-containing syringe). The mixture was subsequently dialyzed overnight against at least ~100 volumes of 1× phosphate buffered saline, pH 7.4 using Spectro/Por™ dialysis membranes (molecular weight cut-off 12 000-14 000 Da). The LNPs were concentrated as required with an Amicon Ultra™ 100 000 MWCO (molecular weight cut-off), regenerated cellulose concentrator.

Encapsulation efficiency was calculated by determining unencapsulated mRNA content by measuring the fluorescence upon the addition of RiboGreen™ to the mRNA-LNP (Fᵢ) and comparing this value to the total mRNA content that is obtained upon lysis of the LNP by 2% Triton X-100 (Fₜ): % encapsulation=(Fₜ−Fᵢ)/Fₜ×100.

The particle size and polydispersity index (PDI) were characterized using a Zetasizer Nano ZS™

### Flow Cytometry In Vivo Studies

The LNPs at the mRNA concentrations indicated were injected intravenously (i.v.) in mice at a volume using the formula weight of the mouse (in grams)*10 μL. Bone marrow and blood were harvested at the time points indicated after the LNP injections.

The bone marrow was harvested and processed into a single cell suspension. In particular, the mice were anesthetized with 5% isoflurane until reflex was lost and then exposed to CO₂ with 1% air. The marrow was isolated from the femur by centrifugation of the bone for 30 s at 3,810 g and resuspended in FACS buffer (1× sterile PBS (pH 7.4), 2.5 mM ethylenediaminetetraacetic acid (EDTA), 0.05% (w/v) sodium azide (NaN₃), 2% (v/v) heat-inactivated fetal bovine serum (HI-FBS)). The bone marrow was then washed once in FACS buffer before final resuspension.

After isolation, the bone marrow and/or blood cells were stained. One to three million cells were added to a well of 96-well round bottom plates and the volume in each well was increased to 150 μL using FACS buffer. Cells were centrifuged at 484 g at 4° C. for 5 minutes and the liquid was discarded. Subsequently, cells were incubated with Fc block and then a solution containing staining antibodies for 45 minutes. Cells were centrifuged at 484 g at 4° C. for 5 minutes and the liquid was discarded. The volume was increased to 150 μL and cells were centrifuged at 484 g at 4° C. for 5 minutes and the liquid was discarded twice. A volume of 200 μL of eFluor 506 staining dye was added at a 1:500 dilution and cells were incubated for 30 minutes. Cells were centrifuged at 484 g at 4° C. for 5 minutes and the liquid was discarded. The volume was increased to 150 μL and cells were centrifuged at 484 g at 4° C. for 5 minutes and the liquid was discarded twice. Single cells were introduced to a flow cytometer (Cytoflex™, Beckman Coulter™). Single colour set-ups were used to generate the compensation matrix which was applied to all the samples.

The blood was harvested and processed into a single cell suspension. In particular, the mice were anesthetized with 5% isoflurane until reflex was lost and then exposed to CO₂ with 1% air. The blood was harvested via immediate cardiac puncture and added to 0.5 mL of 0.5 M EDTA solution. The blood-EDTA solution was then transferred to 12 mL of pre-warmed 1×RBC Lysis buffer and incubated in a 37° C. water bath for 5 minutes. Cells were then centrifuged at 484 g at 4° C. for 5 minutes and the liquid was discarded. Blood cells were then washed in FACS buffer (1× sterile PBS (pH 7.4), 2.5 mM ethylenediaminetetraacetic acid (EDTA), 0.05% (w/v) sodium azide (NaN₃), 2% (v/v) heat-inactivated fetal bovine serum (HI-FBS)) and resuspended in a final volume of FACS buffer.

The flow cytometry data was analyzed using FlowJo™ version 10 (Becton Dickinson™ & Company (BD)). Corresponding T cell sub-sets were identified based on an appropriate gating scheme.

### Example 1: LNPs with High Neutral Lipid Content and Encapsulating CAR are Efficacious In Vivo

The inventors investigated the ability of LNPs encapsulating mRNA encoding a CAR to express a marker encoded by a CAR mRNA.

The CAR-LNPs investigated (Table 4 below) contained elevated levels of neutral lipid (50 mol %; LNPs B, and C) and were compared against an LNP that contained conventional amounts of neutral lipid (10 mol %; LNP A baseline). As shown below, LNPs with elevated neutral lipid transfected most immune cell sub-types, although monocytes showed the highest levels of transfection.

TABLE 4
LNPs encapsulating CAR investigated for in vivo
efficacy against an effector cell

| | | | | Ionizable |
|---|---|---|---|---|
| | | | | lipid (IL) |
| | Mol | Lipid | | citation |
| | % | composition/ | Molar | (incorporated |
| LNP | DSPC | molar ratios | ratios | by reference) |
| A | 10 | Ionizable lipid 1 | 50:10: | WO 2022/ |
| | | (IL1):DSPC: | 38.5:1.5 | 246571; nor- |
| | | Chol:PEG₂₀₀₀- | | MC3 |
| | | DMG | | |
| B | 50 | Ionizable lipid 2 | 27.4:50: | WO 2024/ |
| | | (IL2):DSPC: | 21.1:1.5 | 130421; |
| | | Chol:PEG₂₀₀₀- | | compound |
| | | DMG | | 27 |
| C | 50 | Ionizable lipid 3 | 27.4:50: | U.S. provisional |
| | | (IL3):DSPC: | 21.1:1.5 | application No. |
| | | Chol:PEG₂₀₀₀- | | 63/664,792; |
| | | DMG | | compound |
| | | | | T6 |

The LNPs of Table 4 were prepared encapsulating the anti-CD19 CAR mRNA cargo (FIG. 1B) to deplete CD19⁺ B cells (target cell) in vivo. In particular, a T7 RNA polymerase 24 drives expression of the CAR from a cassette 22, containing a 5′ untranslated region (UTR) 26, a CD19 CAR 28, a porcine teschovirus 1-2A (P2A) 30, a Thy1.1 marker 32 to assess transfection and an hBeta globin 3′ UTR 34. Thy1.1 was used to identify transfection efficiency in the target cells assessed, namely monocytes and macrophages, T cells, and CD4+/CD8+ T cells. The efficacy of the CAR cell transfected with the LNP to target the B cells was assessed by determining the percentage of B cells alive. Thy1.1 expression and the percentage of alive B cells was assessed at 24 hours post-injection.

The physicochemical data in FIG. 1C show that the LNP sizes, polydispersity index (PDI) and encapsulation percentages of LNPs A-C of Table 4 above were all within acceptable ranges.

As shown in FIGS. 2A, 2B and 2C, the LNPs with elevated neutral lipid showed enhanced transfection of monocytes in the blood relative to the baseline with only 10 mol % neutral lipid. LNP B exhibited the highest transfection as measured by Thy1.1 relative to the baseline LNP. Similar trends with macrophages were observed in the bone marrow (FIGS. 3A-C).

### Example 2: LNPs with High Neutral Lipid Content and Encapsulating CAR are Efficacious In Vivo at Various Doses

A further study was conducted to assess the impact of CAR-LNP dose on transfection of target cells as well as B cell depletion. The CAR LNPs investigated are set forth in Table 5 below and contained elevated levels of neutral lipid (LNPs C and D) and were compared against baseline LNPs that contained conventional amounts of neutral lipid (10 mol %; LNP B baseline) and elevated neutral lipid encapsulating mRNA encoding for luciferase.

TABLE 5
LNPs examined in dose titration studies

| | | | | Ionizable | |
|---|---|---|---|---|---|
| | | | | lipid | |
| | | | | (IL) | |
| | | | | citation | |
| | | Lipid | | (incor- | |
| | Mol | composition/ | | porated | |
| | % | molar | Molar | by | |
| LNP | DSPC | ratios | ratios | reference) | Cargo |
| A | 50 | Ionizable | 27.4/50/ | WO 2022/ | FLuc |
| | | lipid 2 | 21.1/1.5 | 246571; | |
| | | (IL1):DSPC: | | nor-MC3 | |
| | | Chol:PEG₂₀₀₀- | | | |
| | | DMG | | | |
| B | 10 | Ionizable | 50:10: | WO 2022/ | anti- |
| | | lipid 1 | 38.5:1.5 | 246571; | CD19 |
| | | (IL1):DSPC: | | nor-MC3 | CAR |
| | | Chol:PEG₂₀₀₀- | | | mRNA |
| | | DMG | | | cargo |
| C | 50 | Ionizable | 27.4/50/ | WO 2024/ | anti- |
| | | lipid 2 | 21.1/1.5 | 130421; | CD19 |
| | | (IL2):DSPC: | | compound | CAR |
| | | Chol:PEG₂₀₀₀- | | 27 | mRNA |
| | | DMG | | | cargo |
| D | 50 | Ionizable | 27.4/50/ | U.S. | anti- |
| | | lipid 3 | 21.1/1.5 | provisional | CD19 |
| | | (IL3):DSPC: | | application | CAR |
| | | Chol:PEG₂₀₀₀- | | No. | mRNA |
| | | DMG | | 63/664,792; | cargo |
| | | | | compound | |
| | | | | T6 | |

LNPs B, C and D of Table 5 were prepared encapsulating the anti-CD19 CAR mRNA cargo to deplete CD19⁺ B cells (target cell) in vivo as set forth in FIG. 1B. LNP A was an FLuc control encapsulating the mRNA cassette of FIG. 1B except encoding FLuc instead of the anti-CD19 CAR mRNA. The doses examined ranged from 0.3 mg/kg to 2.5 mg/kg for LNPs C and D. The controls (LNPs A and B) were dosed at 1 mg/kg. Thy1.1 expression and B cell depletion was assessed at 24 hours post administration.

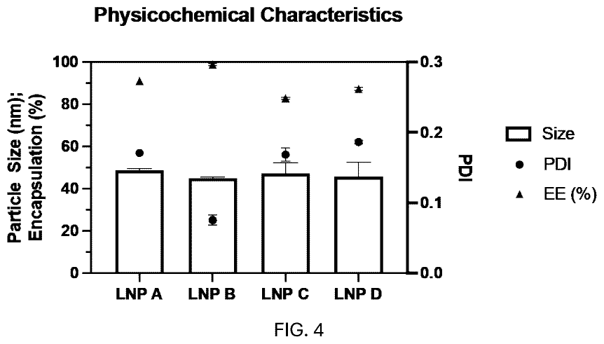

The physicochemical data in FIG. 4 show that the LNP sizes, polydispersity index (PDI) and encapsulation percentages of LNPs A-D of Table 5 above were all within acceptable ranges.

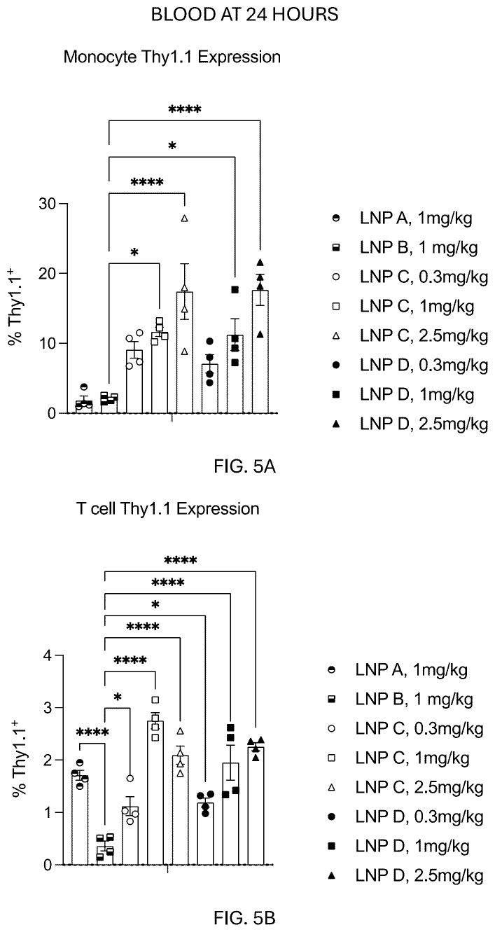

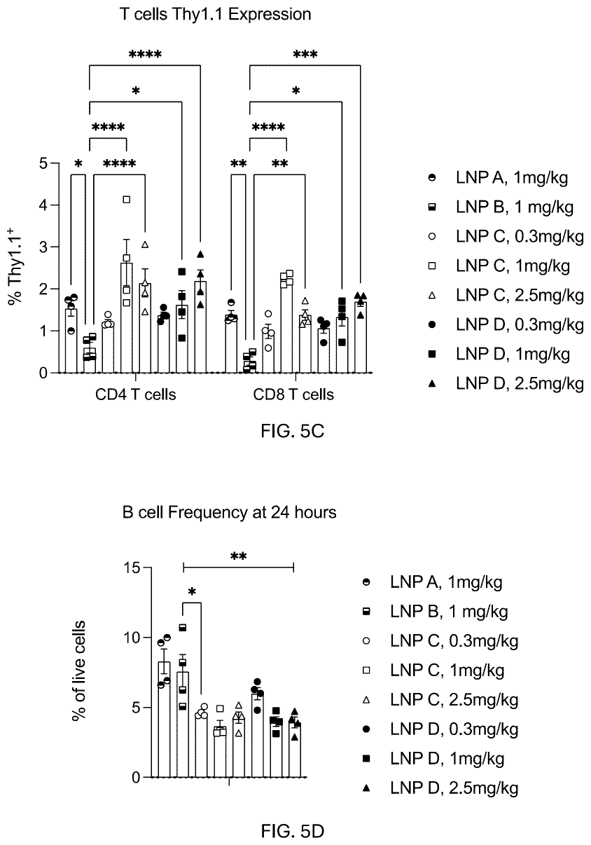

FIGS. 5A-C show that transfection as measured by Thy1.1 in the blood at 24 hours was highest for the LNPs having elevated neutral lipid relative to the controls at most of the doses examined. FIG. 5D shows B cell depletion at the various doses.

### Example 3: LNPs with High Neutral Lipid Content and Encapsulating CAR are Efficacious In Vivo 48 Hours in the Blood at 1 mg/Kg (Study 2)

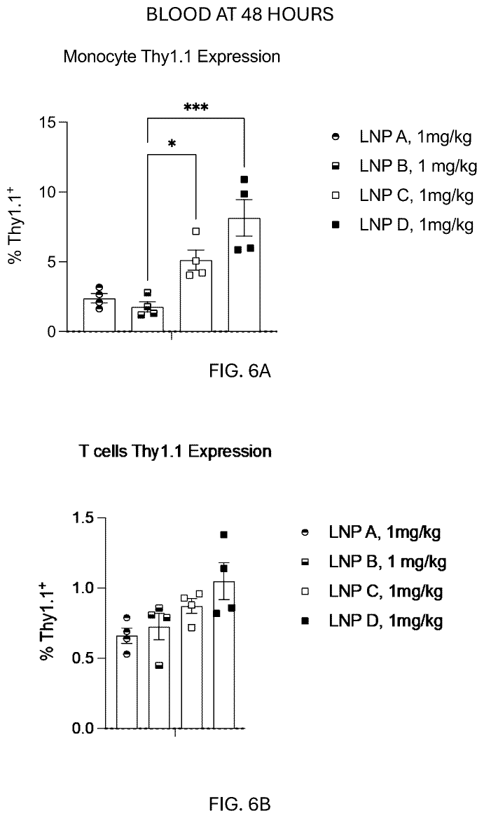

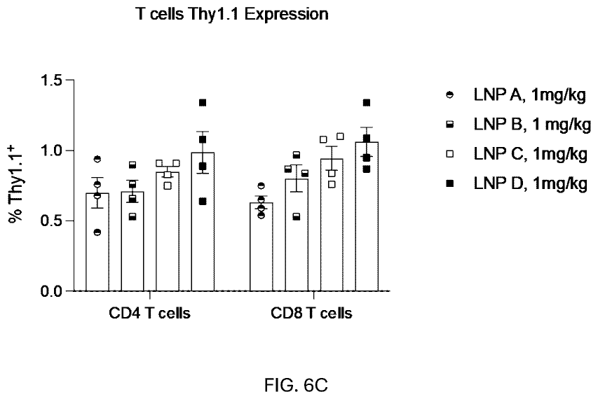

The CAR-LNPs of Table 5 of Example 2 were examined for transfection of immune cell sub-types in the blood at 48 hours post administration and at a dose of 1 mg/kg. FIGS. 6A-C show that transfection as measured by Thy1.1 in the blood at 48 hours was highest for the LNPs having elevated neutral lipid and particularly elevated in monocyte populations.

### Example 4: Target Cell Depletion Time Course Study

In view of the favourable results at 24 and 48 hours, time course experiments were next carried out to assess target cell (B cell) depletion after injection of CAR-LNPs with and without elevated neutral lipid content. The LNPs analyzed are set out in Table 6 below and were prepared as described in the Materials and Methods and as per Example 1 above.

TABLE 6
LNPs assessed for target cell depletion studies

| | | | | Ionizable | |
|---|---|---|---|---|---|
| | | | | lipid | |
| | | | | (IL) | |
| | | | | citation | |
| | | Lipid | | (incor | |
| | Mol | composition/ | | porated | |
| | % | molar | Molar | by | |
| LNP | DSPC | ratios | ratios | reference) | Cargo |
| A | 10 | Ionizable | 50:10: | WO 2022/ | anti- |
| | | lipid 1 | 38.5:1.5 | 246571; | CD19 |
| | | (IL1):DSPC: | | nor-MC3 | CAR |
| | | Chol:PEG₂₀₀₀- | | | mRNA |
| | | DMG | | | cargo |
| B | 50 | Ionizable | 27.4/50/ | WO 2024/ | anti- |
| | | lipid 2 | 21.1/1.5 | 130421; | CD19 |
| | | (IL2):DSPC: | | compound | CAR |
| | | Chol:PEG₂₀₀₀- | | 27 | mRNA |
| | | DMG | | | cargo |

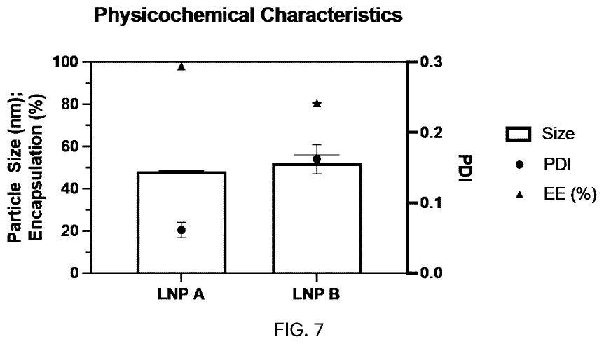

The physicochemical properties of the CAR-LNPs of Table 6 were all within acceptable parameters (FIG. 7).

The LNP dose administered to mice was 0.1 mg/kg and blood samples were withdrawn at 6, 24 and 48 hours post administration. The cargo was anti-CD19 CAR mRNA cargo (FIG. 1B) and PBS was also used as a control.

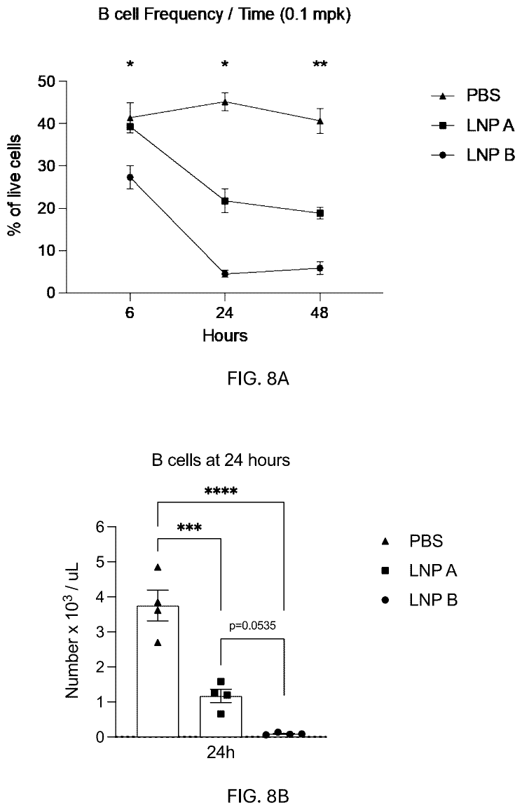

LNP B with elevated neutral lipid depleted B cells (lowest percent of live cells) to a greater extent than LNP A with 10 mol % neutral lipid throughout the time course of 6 to 48 hours (FIG. 8A). The B cell count results at 24 hours are shown in FIG. 8B, which is the time point at which the difference in B cell count between the two LNPs tested was the greatest.

1. A method for delivery of nucleic acid encoding a chimeric antigen receptor (CAR) for expression by an immune cell selected from at least a monocyte or macrophage to bind a receptor on a target cell or molecule in vivo, the method comprising contacting a lipid nanoparticle encapsulating the nucleic acid encoding the CAR with the immune cell ex vivo or in vivo, thereby causing cellular uptake of the nucleic acid to cause the immune cell to express the CAR, the lipid nanoparticle having between 20 mol % and 70 mol % of a neutral or zwitterionic amphipathic lipid having a net-neutral charge at physiological pH, an ionizable cationic lipid, and optionally a sterol, wherein the lipid nanoparticle is substantially uncharged at physiological pH and has an apparent pKa of between 6.0 and 7.5, wherein the immune cell expressing the CAR provides a therapeutic, prophylactic or ameliorative effect in vivo.

2. The method of claim 1, wherein the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

3. The method of claim 1, wherein the lipid nanoparticle comprises at least 25 mol % of the neutral or zwitterionic amphipathic lipid.

4. The method of claim 1, wherein the contacting is in vivo and the immune cell to which the CAR is delivered is present systemically in a subject.

5. The method of claim 4, wherein the contacting is in at least the blood, lymph nodes, spleen or bone marrow.

6. The method of claim 1, wherein the modified immune cell is part of a plurality of modified immune cells that include the macrophage or monocyte and one or more of a dendritic cell, T cell, neutrophil, basophil, eosinophil, mast cell, natural killer cell, myeloid cell, monocyte, lymphoid cell or a combination thereof.

7. The method of claim 6, wherein the plurality of modified immune cells comprises a T cell.

8. The method of claim 1, wherein the nucleic acid is mRNA or vector DNA for expressing an endogenous or exogenous protein, polypeptide or peptide in the immune cell.

9. The method of claim 1, wherein the lipid nanoparticle is for treating a disease or disorder that is an immunological disease or disorder.

10. The method of claim 1, wherein the lipid nanoparticle is for treating a disease or disorder that is a cancer.

11. The method of claim 10, wherein the cancer is a haematological cancer.

12. The method of claim 1, wherein the lipid nanoparticle is for reducing a B cell count in a blood compartment of the subject.

13. The method of claim 1, when the lipid nanoparticle further produces a modified T cell that expresses the CAR.

14. A lipid nanoparticle comprising an encapsulated nucleic acid encoding a chimeric antigen receptor (CAR) for ex vivo or in vivo delivery to an immune cell that is at least a monocyte or macrophage to produce a modified immune cell expressing the CAR, the lipid nanoparticle having between 20 mol % and 70 mol % of a neutral lipid or zwitterionic amphipathic lipid having a neutral or net-neutral charge at physiological pH, an ionizable cationic lipid, and optionally a sterol, wherein the lipid nanoparticle is substantially uncharged at physiological pH and has an apparent pKa of between 6.0 and 7.5.

15. The lipid nanoparticle of claim 14, wherein the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

16. The lipid nanoparticle of claim 14, wherein the lipid nanoparticle is for contacting is in vivo and the immune cell to which the CAR is delivered is present systemically in a subject.

17. The lipid nanoparticle of claim 14, wherein the lipid nanoparticle is for modifying the immune cell in vivo in a subject's blood, lymph nodes, spleen or bone marrow.

18. The lipid nanoparticle of claim 14, wherein the lipid nanoparticle comprises at least 25 mol % of the neutral or zwitterionic amphipathic lipid.

19. The lipid nanoparticle of claim 14, wherein the modified immune cell is part of a plurality of immune cells that include at least the macrophage or monocyte and one or more or a dendritic cell, T cell, neutrophil, basophil, eosinophil, mast cell, natural killer cell, myeloid cell, monocyte, lymphoid cell or a combination thereof.

20. (canceled)

21. (canceled)

22. (canceled)

23. (canceled)

24. (canceled)

25. An ex vivo immune cell preparation comprising the lipid nanoparticle of claim 14.

26. The immune cell preparation of claim 25 comprising immune cells obtained from a human subject.
